SEQUENCE LISTING (4 sequences)

SEQ ID NO: 1 - DNA, 20 bp, Artificial Sequence
  Synthetic construct
agagggagacgtctccgcct

SEQ ID NO: 2 - DNA, 20 bp, Artificial Sequence
  Synthetic construct
agagtctctcagctggtaca

SEQ ID NO: 3 - DNA, 19 bp, Artificial Sequence
  Synthetic construct
cagggttctggatatctgt

SEQ ID NO: 4 - DNA, 20 bp, Artificial Sequence
  Synthetic construct
ctggatatctgtgggacaag

(19) United States
(12) Patent Application Publication
(10) Pub. No.: US 2024/0301403 A1
(43) Pub. Date: Sep. 12, 2024

(54) METHOD FOR MAKING CAR-T LIBRARIES

(71) Applicant: The Regents of the University of California, Oakland, CA (US)

(72) Inventors: Daniel B. GOODMAN, San Francisco, CA (US); Camillia S. AZIMI, San Francisco, CA (US); Kole T. ROYBAL, San Francisco, CA (US); Jeffrey A. BLUESTONE, San Francisco, CA (US); Alexander MARSON, San Francisco, CA (US)

(21) Appl. No.: 18/263,366

(22) PCT Filed: Jan. 28, 2022
(86) PCT No.: PCT/US22/14436

§ 371 Date: Jul. 28, 2023

Related U.S. Application Data
(60) Provisional application No. 63/143,424, filed on Jan. 29, 2021.

(51) Int. Cl. C12N 15/10; C12N 15/86

(52) U.S. Cl. CPC C12N 15/1093; C12N 15/86; C12N 2750/14143

(57) ABSTRACT

This disclosure generally relates to high-throughput and modular methods and compositions for barcode-trackable cellular receptor design, manufacture, and measurement.

### CROSS-REFERENCE TO RELATED APPLICATION

This application claims the benefit of U.S. Provisional Patent Application No. 63/143,424, filed on Jan. 29, 2021, and entitled “METHOD FOR MAKING CAR-T LIBRARIES,” the entirety of which is incorporated by reference.

### INCORPORATION OF THE SEQUENCE LISTING

This application contains a Sequence Listing, which is hereby incorporated herein by reference in its entirety. The contents of the electronic sequence listing 2024-03-07 Sequence_Listing_ST25 048536-673N01US.txt; Size: 1,192 bytes; and Date of Creation: Mar. 7, 2024.

### FIELD

This disclosure generally relates to high-throughput and modular methods and compositions for barcode-trackable cellular receptor design, manufacture, and measurement.

### BACKGROUND

New synthetic cellular receptors (including CARs, SynNotch, etc.) are ushering in many new treatment options for previously intractable cancers, and present promising therapeutic strategies for other diseases. However, the enormous space of possible synthetic receptor designs has only begun to be explored. For example, while CAR T cell therapy has had an unprecedented impact for specific hematological malignancies, current CAR T cell therapies have had limited efficacy in solid tumors.

In order to identify new synthetic receptors which have improved characteristics for various therapeutic purposes, more efficient and high-throughput strategies are required. The present disclosure describes high-throughput and modular methods and compositions for barcode-trackable cellular receptor design, manufacture, and measurement.

Cellular signaling receptors are surprisingly modular. What makes chimeric antigen receptors (CARs) work is the diverse immunological signaling domains can be linearly composed and cross-linked with orthogonal ligands. Signaling through many immune receptors is controlled by short, conserved linear amino acid motifs which bind other proteins. The composition of these linear motifs serve to modulate downstream pathways and drive T-cell function and phenotype. A large proportion of human transmembrane proteins have short unstructured cytoplasmic C-terminal domains with conserved motifs. The compact size and unstructured nature of these endodomains allows them to be readily synthesized at scale with pooled oligo libraries.

### SUMMARY

In certain embodiments, a signaling domain oligonucleotide is provided. The signaling domain oligonucleotide includes, from 5′ to 3′: (a) Type IIs restriction site for a first Type IIs restriction enzyme; (b) forward primer sequence; and (c) a module selected from (i) a module including a signaling domain barcode, dual Type IIs restriction sites for a second Type IIs restriction enzyme, and a signaling domain sequence; and (ii) a module including a signaling domain sequence, dual Type IIs restriction sites for a second Type IIs restriction enzyme, and a signaling domain barcode. The signaling domain oligonucleotide further includes an additional Type IIs restriction site for the first Type IIs restriction enzyme, and a reverse primer sequence. At least one of the forward primer sequence and the reverse primer sequence includes at least one Type IIs restriction site. The Type IIs restriction sites for the first Type IIs restriction enzyme and the second Type IIs restriction enzyme are not identical.

In some embodiments, the signaling domain barcode does not contain a restriction site for either the first Type IIs restriction enzyme or the second Type IIs restriction enzyme. In some embodiments, the signaling domain barcode indicates the identity of the signaling domain sequence. In some embodiments, the signaling domain barcode includes no more than about 3 consecutive G or C bases. In some embodiments, the signaling domain barcode includes no more than about 4 consecutive A or T bases. In some embodiments, the signaling domain barcode includes a GC content that is at least about 30% and no more than about 70%. In some embodiments, the signaling domain sequence does not contain a restriction site for either the first Type IIs restriction enzyme or the second Type IIs restriction enzyme. In some embodiments, the signaling domain sequence includes one or more sequences each independently derived from a sequence encoding an intracellular portion of a membrane protein. In some embodiments, each of the one or more sequences in the signaling domain sequence is at least 80% identical to the sequence encoding the intracellular portion of the membrane protein. In some embodiments, the membrane protein includes a class 1 human membrane protein, a class 3 human membrane protein, or a membrane protein from a species that is capable of infecting a mammalian cell.

In some embodiments, the membrane protein is selected from the list consisting of CD28, ICOS, CTLA4, PD1, PD1H, BTLA, B71, B7H1, CD226, CRTAM, TIGIT, CD96, TIM1, TIM2, TIM3, TIM4, CD2, SLAM, 2B4, Ly108, CD84, Ly9, CRACC, BTN1, BTN2, BTN3, LAIR1, LAG3, CD160, 4-1BB, OX40, CD27, GITR, CD30, TNFR1, TNFR2, HVEM, LT_R, DR3, DCR3, FAS, CD40, RANK, OPG, TRAILR1, TACI, BAFFR, BCMA, TWEAKR, EDAR, XEDAR, RELT, DR6, TROY, NGFR, CD22, SIGLEC-3, SIGLEC-5, SIGLEC-7, KLRG1, NKR-P1A, ILT2, KIR2DL1, KIR3DL1, CD94-NKG2A, CD300b, CD300e, TREM1, TREM2, ILT7, ILT3, ILT4, TLT-1, CD200R, CD300a, CD300f, DC-SIGN, B7-2, Allergin-1, LAT, BLNK, LAYN, SLP76, EMB-LMP1, HIV-NEF, HVS-TIP, HVS-ORF5, and HVS-stpC. In some embodiments, the membrane protein is selected from the list consisting of OX40, ICOS, 4-1BB, CTLA4, CD28, CD30, CD2, CD27, and CD226.

In some embodiments, the signaling domain sequence includes more than one sequence, each derived from a sequence encoding an intracellular portion of a different membrane protein. In some embodiments, the signaling domain sequence includes one or more mutations. In some embodiments, the one or more mutations include phosphorylation and ubiquitination knockouts, deletion scans, constitutive phosphorylation mimics, known human mutations, and deep mutational scan. In some embodiments, the signaling domain sequence encodes a peptide that can no longer be modified by phosphorylation or ubiquitination. In some embodiments, all codons for tyrosine, serine, and threonine residues in the signaling domain sequence are replaced with codons for aspartate acid. In some embodiments, the first Type IIs restriction enzyme and the second Type IIs restriction enzyme are independently selected from the group consisting of BsaI, BsmBI, Esp3I, BbsI, NotI, and PspXI. In some embodiments, the signaling domain oligonucleotide is synthetic. In some embodiments, the signaling domain oligonucleotide further includes a Unique Molecular Identifier (UMI).

In another, interrelated aspect, a collection of signaling domain oligonucleotides including a plurality of the signaling domain oligonucleotides described herein, including embodiments, is provided. In some embodiments, each one of the signaling domain oligonucleotides has a unique pair of the forward primer sequence and the reverse primer sequence.

In another, interrelated aspect, a method for preparing a plurality of receptor oligonucleotides is provide. The method includes assembling the collection of signaling domain oligonucleotides described herein with a plurality of cloning vectors to produce a plurality of intermediate vectors, wherein a member of the plurality of cloning vectors includes, from 5′ to 3′, dual Type IIs restriction sites for a first Type IIs restriction enzyme, an immune cell-activating domain, an autoproteolytic peptide sequence, and a marker protein sequence. The method further includes assembling the plurality of intermediate vectors with a plurality of additional vectors, wherein a member of the plurality of additional vectors includes dual Type IIs restriction sites for a second Type IIs restriction enzyme, an extracellular domain (ECD), sequence and a transmembrane domain, thereby producing the plurality of receptor oligonucleotides.

In another, interrelated aspect, a method for preparing a plurality of receptor oligonucleotides is provided. The method includes assembling the collection of signaling domain oligonucleotides as described herein with a plurality of cloning vectors to produce a plurality of intermediate vectors, wherein a member of the plurality of cloning vectors includes, a 5′ homology arm sequence, an extracellular domain (ECD) sequence, a transmembrane domain, dual Type IIs restriction sites for a first Type IIs restriction enzyme, and a 3′ homology arm sequence. The method further includes assembling the plurality of intermediate vectors with a plurality of additional vectors, wherein a member of the plurality of additional vectors includes dual Type IIs restriction sites for a second Type IIs restriction enzyme, an immune cell-activating domain, an autoproteolytic peptide sequence, and a marker protein sequence, thereby producing the plurality of receptor oligonucleotides.

In some embodiments, the ECD sequence encodes an extracellular antigen-binding domain capable of binding to a target antigen. In some embodiments, the antigen-binding domain includes a single chain variable fragment (scFv). In some embodiments, a member of the plurality of cloning vectors further includes a promoter sequence or an autoproteolytic peptide sequence. In some embodiments, the autoproteolytic peptide sequence encodes a 2A self-cleaving peptide. In some embodiments, one or more of the plurality of receptor oligonucleotides further include a 5′ homology arm and a 3′ homology arm. In some embodiments, the method further includes assembling a plurality of vectors including guide RNA (gRNA) sequences with the plurality of cloning vectors. In some embodiments, the assembling steps are conducted concurrently; and wherein the method does not use polymerase chain reaction (PCR). In some embodiments, the assembling steps are conducted using Type IIs restriction enzymatic reactions. In some embodiments, each of the sequences is identifiable by a barcode. In some embodiments, the assembling steps are conducted combinatorially. In some embodiments, the method further includes incorporating a Unique Molecular Identifier (UMI) in each member of the plurality of receptor oligonucleotides.

In another, interrelated aspect, a plurality of receptor oligonucleotides produced by the methods described herein, including embodiments, is provided.

In some embodiments, a member of the plurality of receptor oligonucleotides includes, in a 5′ to 3′ direction: (a) a unique molecular identifier (UMI), (b) an extracellular antigen-binding domain sequence, (c) a transmembrane domain, (d) a signaling domain sequence, (e) an immune cell-activating domain, and (f) an autoproteolytic peptide sequence. In some embodiments, a member of the plurality of receptor oligonucleotides further includes a promoter or an autoproteolytic peptide sequence between the UMI and the ECD sequence. In some embodiments, a member of the plurality of receptor oligonucleotides further includes a gRNA sequence 5′ to the UMI. In some embodiments, a member of the plurality of receptor oligonucleotides includes, in a 5′ to 3′ direction: (a) a 5′ homology arm sequence, (b) an extracellular antigen-binding domain sequence, (c) a transmembrane domain, (d) a signaling domain sequence, (e) an immune cell-activating domain, (f) an autoproteolytic peptide sequence, (g) a unique molecular identifier (UMI), and (h) a 3′ homology arm sequence. In some embodiments, a member of plurality of receptor oligonucleotides encodes a chimeric antigen receptor.

In another, interrelated aspect, a vector including the signaling domain oligonucleotides described herein, including embodiments, is provided.

In another, interrelated aspect, a collection of vectors including the collection of signaling domain oligonucleotides described herein, including embodiments, is provided.

In another, interrelated aspect, a plurality of vectors including the plurality of receptor oligonucleotides described herein, including embodiments, is provided.

In another, interrelated aspect, the vector, the collection of vectors, or the plurality of vectors described herein, including embodiments, further includes a plurality of restriction enzyme cleavage sites, wherein the restriction enzyme cleavage sites are recognized by 1, 2, or 3 different selected restriction enzymes; and wherein the restriction enzyme cleavage sites are spaced throughout the plasmid no more than about 1,000 bp apart, such that digestion with the selected restriction enzyme(s) produces plasmid fragments no more than about 1,000 bp in length.

In some embodiments, the restriction enzyme cleavage sites are spaced throughout the plasmid no more than about 500 bp apart, such that digestion with the selected restriction enzyme(s) produces plasmid fragments no more than about 500 bp in length. In some embodiments, the selected restriction enzymes includes BsaI, BsmBI, Esp3I, BbsI, NotI, and PspXI. In some embodiments, the vector is a plasmid competent for replication in a host cell.

In another, interrelated aspect, a recombinant immune cell, including a member of the receptor oligonucleotides described herein, including embodiments, is provided. The recombinant immune cell is capable of expressing a chimeric receptor encoded by the member of the receptor oligonucleotide.

In some embodiments, the recombinant immune cell is a recombinant T cell. In some embodiments, the recombinant T cell is a recombinant CD4+ T cell or a recombinant CD8+ T cell. In some embodiments, the recombinant immune cell exhibits one or more improved properties including: cellular proliferation; resistance to immune cell exhaustion; expression of cytokines; sensitivity to antigen; specificity to antigen; stability of differentiation state; trafficking to a specific tissue or organ in vivo; ability to kill target cells; or differentiation into a desired state.

In another, interrelated aspect, a collection of the recombinant immune cells described herein, including embodiments, is provided. Each member of the collection of the recombinant immune cells is identifiable by a UMI.

In another, interrelated aspect, a method of preparing the recombinant immune cell described herein is provided. The method includes (a) providing a recombinant immune cell capable of protein expression and (b) contacting the provided cell with one or more members of the plurality of receptor oligonucleotides described herein, including embodiments.

In another, interrelated aspect, a method for identifying a signaling domain sequence that modulates a designated property of a cell under specified conditions is provided. The method includes: providing the recombinant immune cell described herein or the collection of recombinant immune cells described herein; applying the specified conditions to the recombinant immune cell or the collection of recombinant immune cells; identifying the recombinant cells that exhibit the designated property; and/or identifying the signaling domain sequence in the recombinant cells that exhibit the designated property.

In some embodiments, the specified conditions include interaction with a target antigen. In some embodiments, the designated property includes one or more of the following: cellular proliferation; resistance to immune cell exhaustion; expression of cytokines; sensitivity to antigen; specificity to antigen; stability of differentiation state; trafficking to a specific tissue or organ; ability to kill target cells; or differentiation into a desired state. In some embodiments, the identification is performed in vitro or in vivo. In some embodiments, the identification is performed by single-cell sequencing, fluorescence-activated cell sorting (FACS), bead-based enrichment, or enrichment in a population of mixed cells over time after a stimulus.

### BRIEF DESCRIPTION OF THE DRAWINGS

FIGS. 1A-1B show an exemplary illustrations of the 5′ cloning system. The barcode was placed upstream of the promoter, so that the promoter, scFv, and transmembrane regions of the receptor oligonucleotide were inserted into the internal restriction site region. The COSTIM domain corresponds to the signaling domain sequence.

FIG. 2 shows exemplary illustrations of the 3′ cloning system. The barcode was placed downstream of the signaling domain sequence, so that the CD3ζ domain, the 2A peptide, a marker protein, and a final polyA/2A peptide and a barcode were inserted into the internal restriction site region. The COSTIM domain corresponds to the signaling domain sequence.

FIG. 3 shows an exemplary schematic illustration of the process for making the oligonucleotide encoding the chimeric receptor using the 3′ cloning system and AAV-based vectors. The Costim domain corresponds to the signaling domain sequence.

FIG. 4 shows an exemplary schematic illustration of the construction of the CAR library.

### DETAILED DESCRIPTION

As described in greater detail below, the present disclosure generally relates to compositions and methods for preparing a plurality of oligonucleotides in a high-throughput and highly modular manner. The compositions provided in the present disclosure generally relates to oligonucleotides that encode chimeric signaling domains of transmembrane proteins and polypeptides. The compositions provided herein also encompass oligonucleotides that encode chimeric receptors (CRs). In some embodiments, the oligonucleotides provided herein encode chimeric antigen receptors (CARs). Additionally, the present disclosure encompasses vectors and recombinant and/or engineered cells comprising the compositions. Further, the present disclosure also encompasses methods, platforms, system, and kits for preparing and/or using the compositions.

In some embodiments, the present disclosure provides a signaling domain oligonucleotide. In some embodiments, the signaling domain oligonucleotide comprises multiple domains and sequences, including without limitation, signaling domain sequences. In some embodiments, each of the domains and sequences in the signaling domain oligonucleotide is associated with a barcode. In certain embodiments, the signaling domain oligonucleotide also comprises a unique molecular identifier (UMI). In other aspects, the present disclosure provides methods for preparing a plurality of receptor oligonucleotides by cloning the signaling domain oligonucleotide into one or more suitable vectors. Also provided herein are receptor oligonucleotides produced by the methods provided herein. In additional embodiments, the present disclosure encompasses methods for identifying, or screening, a signaling domain sequence that modulates a designated property of a cell under specified conditions.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In some embodiments, the genes, nucleic acid sequences, amino acid sequences, peptides, polypeptides and proteins are human. The term “gene” is also intended to include variants thereof.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

A “library” as used herein refers to a system or set of a plurality of different oligonucleotides, including without limitation, the signaling domain oligonucleotides and/or the receptor oligonucleotides provided herein, or a system or set of engineered cells expressing a plurality of different CRs and/or CARs, wherein the different CRs and/or CARs have different signaling domains. In a pooled library, the plurality of different oligonucleotides, or engineered cells are present as a pool in the same container. In an arrayed library, the plurality of different oligonucleotides, or engineered cells are present in separate or individual containers.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

The singular form “a”, “an”, and “the” include plural references unless the context clearly dictates otherwise. For example, the term “a cell” includes one or more cells, comprising mixtures thereof.

The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### Signaling Domain Oligonucleotides

In some embodiments, the present disclosure provides a signaling domain oligonucleotide. The term “domain” as used herein, in the context of sequence modules in an oligonucleotide, refers to the nucleotide sequence encoding a protein domain. A protein domain as used herein can be either a structured segment of a polypeptide having one or more biological functions, or an unstructured segment of a polypeptide that retain one or more biological functions. For example, structured segments of a polypeptide may encompass, but is not limited to, a continuous or discontinuous plurality of amino acids, or portions thereof, in a folded polypeptide that comprise a three-dimensional structure which contributes to a particular function of the polypeptide. In other instances, a protein domain may include an unstructured segment of a polypeptide comprising two or more amino acids which maintains a particular function of the polypeptide. Also encompassed within this definition are protein domains that may be disordered or unstructured but become structured or ordered upon association with a target or binding partner.

In some embodiments, the signaling domain oligonucleotide comprises, from 5′ to 3′, one or more of the following components: a) a Type IIS restriction site for a first Type IIS restriction enzyme; b) a forward primer sequence; c) a module comprising a signaling domain barcode, dual Type IIS restriction sites for a second Type IIS restriction enzyme, and a signaling domain sequence; d) an additional Type IIS restriction site for the first Type IIS restriction enzyme; and e) a reverse primer sequence.

In other embodiments, the signaling domain oligonucleotide comprises, from 5′ to 3′, one or more of the following components: a) a Type IIS restriction site for a first Type IIS restriction enzyme; b) a forward primer sequence; c) a module comprising a signaling domain sequence, dual Type IIS restriction sites for a second Type IIS restriction enzyme, and a signaling domain barcode; d) an additional Type IIS restriction site for the first Type IIS restriction enzyme; and e) a reverse primer sequence.

The term “barcode” as used herein refers to a contiguous nucleic acid segment or two or more non-contiguous nucleic acid segments that function as an identifier that conveys or is capable of conveying information. In some embodiments, the barcode is part of a larger nucleic acid segment. In some embodiments, the barcode is adjacent to another nucleic acid segment which the barcode identifies. In an exemplary embodiment, the barcode is a signaling domain barcode, i.e., the barcode identifies a particular signaling domain sequence.

In certain embodiments, at least one of the forward primer sequence and the reverse primer sequence comprises a Type IIS restriction site. In certain embodiments, the Type IIS restriction sites for the first Type IIS restriction enzyme and the second Type IIS restriction enzyme are not identical. In some embodiments, the signaling domain oligonucleotide is double-stranded. In other embodiments, the signaling domain oligonucleotide is single-stranded. In some specific embodiments, the signaling domain oligonucleotide is double-stranded and comprises about 100 base pairs to about 2000 base pairs. In other specific embodiments, the signaling domain oligonucleotide is double-stranded and comprises about 200 base pairs to about 500 base pairs. In one exemplary embodiment, the signaling domain oligonucleotide is double-stranded and comprises about 230 base pairs. In certain embodiments, about 165 bp (i.e., about 55 amino acids) of the 230 base pairs are used for the costimulatory domain codons, i.e., the signaling domain sequence in the transmembrane domain oligonucleotides described herein. In other exemplary embodiments, the signaling domain oligonucleotide is double-stranded and comprises more than about 300 base pairs. In certain embodiments, at least a portion of the more than about 300 base pairs can be used for the costimulatory domain codons. In other embodiments, all of the more than about 300 base pairs can be used for the costimulatory domain codons.

In some embodiments, the signaling domain barcode does not contain a restriction site for either the first Type IIS restriction enzyme or the second Type IIS restriction enzyme. In some embodiments, the signaling domain barcode indicates the identity of the signaling domain sequence. In some embodiments, the signaling domain barcode is double-stranded and comprises about 5 base pairs to about 30 base pairs. In other embodiments, the signaling domain barcode is double-stranded and comprises about 6 base pairs to about 25 base pairs. In some embodiments, the signaling domain barcode is double-stranded and comprises about 7 base pairs to about 20 base pairs. In other embodiments, the signaling domain barcode is double-stranded and comprises about 8 base pairs to about 18 base pairs. In an exemplary embodiment, the signaling domain barcode is double-stranded and comprises 9 base pairs. In another exemplary embodiment, the signaling domain barcode is double-stranded and comprises 10 base pairs. In yet another exemplary embodiment, the signaling domain barcode is double-stranded and comprises 15 base pairs. In some embodiments, the signaling domain barcode comprises synonymous codons.

In some embodiments, the signaling domain barcode comprises no more than 3 consecutive G or C bases. In other embodiments, the signaling domain barcode comprises no more than 4 consecutive A or T bases. In some embodiments, the signaling domain barcode comprises a GC content that is at least about 30% and no more than about 70%. In other embodiments, the signaling domain barcode does not contain a restriction site for either the first Type IIS restriction enzyme or the second Type IIS restriction enzyme. In some embodiments, the signaling domain sequence does not contain a restriction site for either the first Type IIS restriction enzyme or the second Type IIS restriction enzyme.

A Type IIS restriction enzyme as used herein refers to the subtype of Type II restriction enzymes which recognizes asymmetric DNA sequences and cleave at a defined distance outside of its recognition sequence. For example, certain Type IIS restriction enzymes cleave at a distance within 1 to 20 nucleotides of its recognition sequence. Thus, Type IIS restriction enzymes are commonly used for sequence-independent cloning strategies. In some embodiments, the first Type IIS restriction enzyme and the second Type IIS restriction enzyme are independently selected from BsaI, BsmBI, Esp3I, BbsI, NotI, and PspXI. In some embodiments, the first Type IIS restriction enzyme and/or the second Type IIS restriction enzyme are Esp3I. However, it will be appreciated by a skilled person in the art that any Type IIS restriction enzymes or any restriction enzymes with the same mechanism of action can be used.

In some embodiments, the signaling domain sequence comprises one or more sequences each independently derived from a sequence encoding an intracellular portion of a membrane protein. In some embodiments, each of the one or more sequences in the signaling domain sequence is at least 70%, 75%, 80%, 85%, 90%, or 99% identical to a sequence encoding an intracellular portion of a membrane protein. In some embodiments, each of the one or more sequences in the signaling domain sequence is 100% identical to a sequence encoding an intracellular portion of a membrane protein.

In some embodiments, the membrane protein comprises a class 1 human membrane protein, a class 3 human membrane protein. For example, in some embodiments, the membrane protein comprises a protein that is categorized by UniProt as a class 1 or a class 3 human membrane protein. One skilled in the art would understand how to obtain the information on such proteins through UniProt's database.

In some embodiments, the membrane protein comprises a membrane protein from a species that is capable of infecting a mammalian cell. In certain exemplary embodiments, the membrane protein can be selected from CD28, ICOS, CTLA4, PD1, PD1H, BTLA, B71, B7H1, CD226, CRTAM, TIGIT, CD96, TIM1, TIM2, TIM3, TIM4, CD2, SLAM, 2B4, Ly108, CD84, Ly9, CRACC, BTN1, BTN2, BTN3, LAIR1, LAG3, CD160, 4-1BB, OX40, CD27, GITR, CD30, TNFR1, TNFR2, HVEM, LT_R, DR3, DCR3, FAS, CD40, RANK, OPG, TRAILR1, TACI, BAFFR, BCMA, TWEAKR, EDAR, XEDAR, RELT, DR6, TROY, NGFR, CD22, SIGLEC-3, SIGLEC-5, SIGLEC-7, KLRG1, NKR-P1A, ILT2, KIR2DL1, KIR3DL1, CD94-NKG2A, CD300b, CD300e, TREM1, TREM2, ILT7, ILT3, ILT4, TLT-1, CD200R, CD300a, CD300f, DC-SIGN, B7-2, Allergin-1, LAT, BLNK, LAYN, SLP76, EMB-LMP1, HIV-NEF, HVS-TIP, HVS-ORF5, and HVS-stpC. In some embodiments, the membrane protein can be selected from OX40, ICOS, 4-1BB, CTLA4, CD28, CD30, CD2, CD27, and CD226. In other embodiments, the membrane protein can be selected from 2B4, 4-1BB, Allergin-1, B7-1, B7-2, B7H1, BAFFR, BCMA, BLNK, BTLA, BTN1, BTN2, BTN3, CD160, CD2, CD200R, CD22, CD226, CD27, CD28, CD30, CD300a, CD300b, CD300e, CD300f, CD40, CD84, CD96, CRACC, CRTAM, CTLA4, DCR3, DC-SIGN, DR3, DR6, EDAR, FAS, GITR, HIV-NEF, HVEM, HVS-stpC, HVS-TIP, ICOS, ILT2, ILT3, ILT4, ILT7, KIR2DL1, KIR3DL1, KLRG1, LAG3, LAIR1, LAT, LAYN, LT_R (TNFRSF3), Ly108, Ly9, NGFR, NKR-P1A, TNFRSF11B, OX40, PD1, PD1H, RANK, RELT, SIGLEC-3, SIGLEC-5, SIGLEC-7, SLAM, SLP76, TACI, TIGIT, TIM1, TIM2, TIM3, TIM4, TLT-1, TNFR1, TNFR2, TRAILR1, TREM1, TREM2, TROY, TWEAKR, or XEDAR.

In other embodiments, the signaling domain sequence as used herein is not selected from the list consisting of Allergin-1, B7-2, BAFF-R, BTLA, CD160, CD2, CD200R, CD22, CD226, CD244, CD27, CD28, CD300a, CD300b, CD300e, CD300f, CD7, CD72, CD80, CD94, CD96, CRACC, CRTAM, CTLA4, CXAR, DC-SIGN, HAVR, ICOS, ILT2, ILT3, ILT4, ILT7, KIR2DL1, KIR3DL1, KLRG1, LAG3, LAIR1, NC14xSAG, NKG2D, NKR-P1A, NTB-A, OX40, PD1L, PDCD1, Pir-B, SIGLEC-3.7.9, TACI, TIGIT, TIM-1, TLT-1, HVEM, 4-1BB, GITR, DR3, CD40, CD30, TREM1, TREM2, and TRML2.

In some embodiments, the intracellular portion of a membrane protein is the intracellular signal transduction domain of a protein. In some embodiments, the intracellular portion of a membrane protein comprises a CAR costimulatory domain. However, it is understood that the membrane proteins listed here are not exhaustive examples. Any protein or peptide that has an intracellular portion that functions or is conjectured to function as a signaling domain of a membrane protein as described immediately above is contemplated by the present invention.

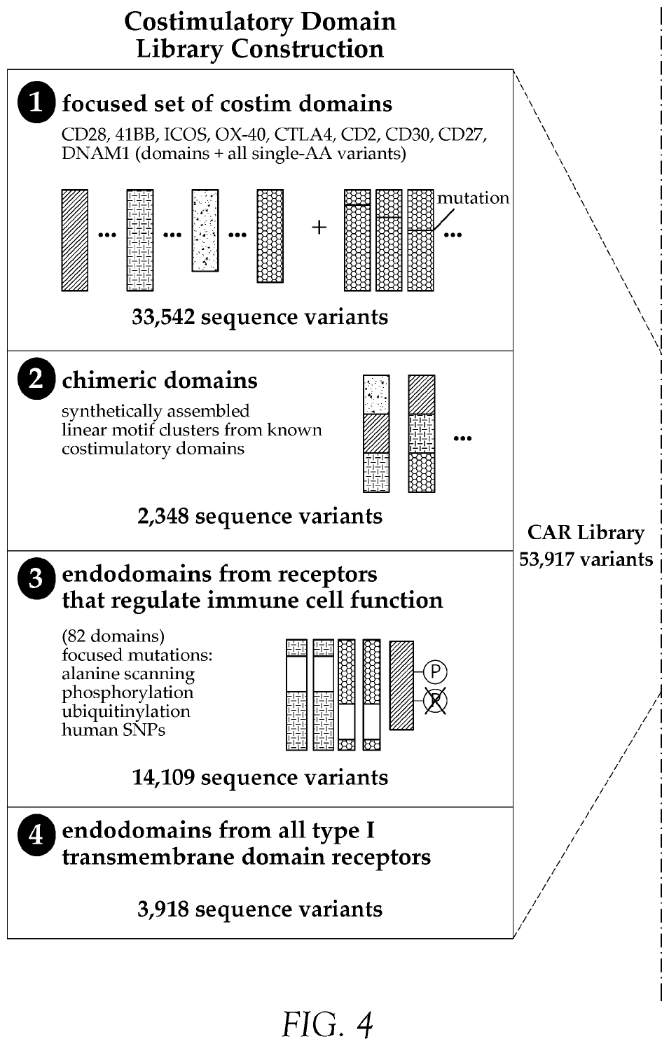

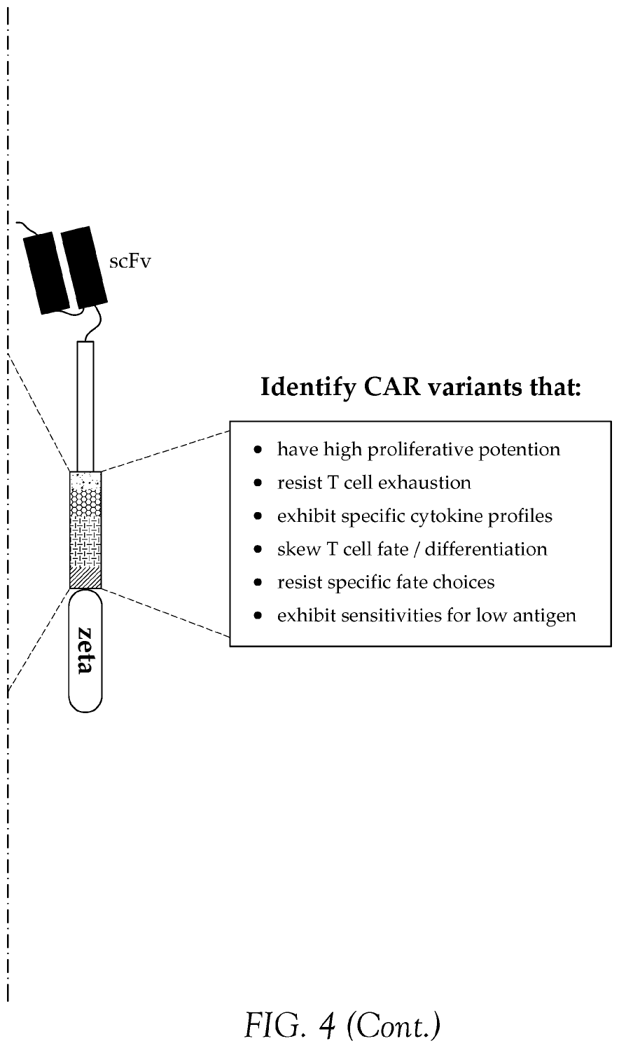

One particular feature of the present disclosure is the modularity of the signaling domain sequences that can be constructed in the signaling domain oligonucleotides provided herein. In some embodiments, the signaling domain sequence comprises more than one sequence and each of the more than one sequence is derived from a sequence encoding an intracellular portion of a different membrane protein. For example, in one embodiment, the signaling domain sequence comprises 2 sequences (chimeric) and each of the 2 sequences is independently derived from a membrane protein encompassed herein. In another exemplary embodiment, the signaling domain sequence comprises 3 or more sequences (multimeric) and each of the 3 or more sequences is independently derived from a membrane protein encompassed herein. An illustration is provided in FIG. 4. As illustrated in FIG. 4, the oligonucleotides comprising various signaling domain sequences or combinations thereof can then be pooled into a library. The library can be subsequently screened for particular signaling domains that exhibit desired features.

In further embodiments of the present disclosure, the signaling domain sequence comprises one or more mutations. The term “mutation” is used as it is in the art and generally means an alteration in the nucleic acid sequence. Such mutations can include any naturally occurring mutations and artificial mutations. In some embodiments, the one or more mutations comprise any known mutations in human. For example, some known human missense mutations can be obtained from ENSEMBL ProtVar database. A skilled person in the art would understand how to obtain such commonly known human mutation information.

In some embodiments, the one or more mutations comprise phosphorylation knockouts, ubiquitination knockouts, or both. In an exemplary embodiment, the signaling domain sequence comprises a sequence in which the codons for one or more of the ubiquitinated residues, phosphorylated residues, or both are replaced with codons for alanine. In such embodiments, the signaling domain sequence encodes a peptide that can no longer be modified by phosphorylation or ubiquitination. In other embodiments, the one or more mutations comprise constitutive phosphorylation mimics. For instance, in some embodiments, all codons for tyrosine, serine, and threonine residues the signaling domain sequence can be replaced with codons for aspartate acid.

In some embodiments, the one or more mutations comprise deletion scans. In an exemplary embodiment, variant chunks can be generated by changing sliding windows of 6 amino acids, moving 3 amino acids at a time, to alanine. In other embodiments, the one or more mutations can be generated by deep mutational scan. In exemplary embodiments, for any of the signaling domain sequences or their encoded proteins disclosed herein, single amino acid insertions, deletions, and changes can be generated for each domain, one group per domain.

In some embodiments, the signaling domain oligonucleotide is synthetic. The oligonucleotide can be synthesized by commonly known methods in the art.

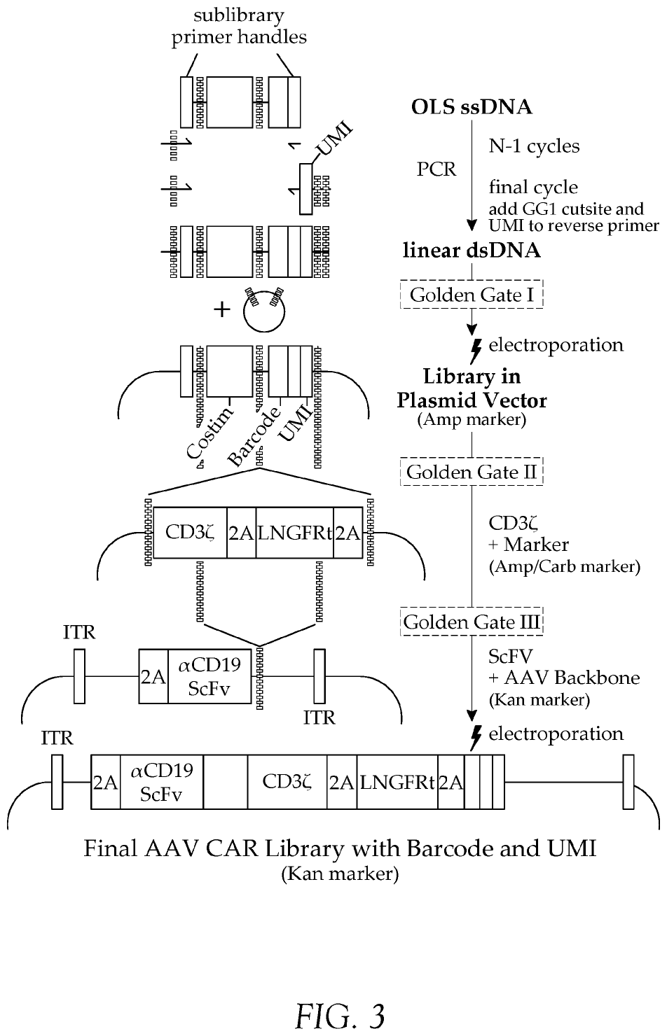

In some embodiments, the signaling domain oligonucleotide further comprises a Unique Molecular Identifier (UMI). The term “unique molecular identifier” as used herein refers to a short nucleic acid sequence added to a nucleic acid molecule to identify the specific molecule. In some embodiments, the UMI is incorporated to the nucleic acid fragment in the last step of the preparation of the signaling domain oligonucleotide. In some embodiments, at the final cycle in preparation of the signaling domain oligonucleotide, an additional reverse primer is added that contains the UMI sequence as well as two additional Type IIs restriction sites, as illustrated in FIG. 3.

Also encompassed by the present disclosure is a collection of signaling domain oligonucleotides comprising a plurality of the signaling domain oligonucleotides disclosed herein. In some embodiments, each one of the signaling domain oligonucleotides has a unique pair of the forward primer sequence and the reverse primer sequence. In some embodiments, the unique pair of the forward primer sequence and the reverse primer sequence can be used to pull a subset of signaling domain oligonucleotides. In one exemplary embodiment, the subset of signaling domain oligonucleotides comprises a contiguous linear portion of a larger signaling domains. In another exemplary embodiment, the subset of signaling domain oligonucleotides comprises concatenated linear portions of multiple signaling domains. In another exemplary embodiment, the subset of signaling domain oligonucleotides share the same family of signaling domain sequences. Table 1 provides a non-exclusive, exemplary list of manually annotated known costimulatory proteins from human and a few from human-associated viruses.

TABLE 1
Exemplary costimulatory proteins from human and human-associated viruses.

| | | | | | | | | Man- | Man- | | |
|---|---|---|---|---|---|---|---|---|---|---|---|
| Super- | | | Co- | | | | In- | ual | ual | Skip | Uniprot |
| family | Family | Protein | signaling | Ligand | Synonyms | Uniprot | clude | start | stop | mut | acc |
| IgRSF | CD28 | CD28 | Stimulatory | B71, B72, | | CD28_HUMAN | 1 | | | 0 | P10747 |
| | | | | B7H2 | | | | | | | |
| | | | | (human) | | | | | | | |
| IgRSF | CD28 | ICOS | Stimulatory | B7H2 | CD278 | ICOS_HUMAN | 1 | | | 0 | Q9Y6W8 |
| IgRSF | CD28 | CTLA4 | Inhibitory | B71, B72, | CD152 | CTLA4_HUMAN | 1 | | | 0 | P16410 |
| | | | | B7H2 | | | | | | | |
| | | | | (human) | | | | | | | |
| IgRSF | CD28 | PD1 | Inhibitory | B7H1, | CD279 | PDCD1_HUMAN | 1 | | | 0 | Q15116 |
| | | | | B7DC | | | | | | | |
| IgRSF | CD28 | PD1H | Inhibitory | Unknown | VISTA | VISTA_HUMAN | 1 | | | 0 | Q9H7M9 |
| IgRSF | CD28 | BTLA | Inhibitory | HVEM, | CD272 | BTLA_HUMAN | 1 | | | 0 | Q7Z6A9 |
| | | | | UL144 | | | | | | | |
| IgRSF | B7 | B71 | Inhibitory | B7H1, | CD80 | CD80_HUMAN | 1 | | | 0 | P33681 |
| | | | | CD28, | | | | | | | |
| | | | | CTLA4 | | | | | | | |
| IgRSF | B7 | B7H1 | Inhibitory | PD1, B71 | CD274, | PD1L1_HUMAN | 1 | | | 0 | Q9NZQ7 |
| | | | | | PDL1 | | | | | | |
| IgRSF | CD226 | CD226 | Stimulatory | CD112, | DNAM1 | CD226_HUMAN | 1 | | | 0 | Q15762 |
| | | | | CD155 | | | | | | | |
| IgRSF | CD226 | CRTAM | Stimulatory | NECL2 | CD355 | CRTAM_HUMAN | 1 | | | 0 | O95727 |
| IgRSF | CD226 | TIGIT | Inhibitory | CD112, | VSIG9, | TIGIT_HUMAN | 1 | | | 0 | Q495A1 |
| | | | | CD113, | VSTM3 | | | | | | |
| | | | | CD155 | | | | | | | |
| IgRSF | CD226 | CD96 | Unclear | CD111, | TACTILE | TACT_HUMAN | 1 | | | 0 | P40200 |
| | | | | CD155 | | | | | | | |
| IgRSF | TIM | TIM1 | Stimulatory | TIM4, | HAVCR1, | HAVRI_HUMAN | 1 | | | 0 | Q96D42 |
| | | | | TIM1, PS | KIM1 | | | | | | |
| IgRSF | TIM | TIM2 | Inhibitory | SEMA4A, | TIMD2 | TIMD2_MOUSE | 1 | | | 0 | Q8R183 |
| | | | | H-ferritin | | | | | | | |
| IgRSF | TIM | TIM3 | Inhibitory | Galectin9, | HAVCR2, | HAVR2_HUMAN | 1 | | | 0 | Q8TDQ0 |
| | | | | PS | KIM3 | | | | | | |
| IgRSF | TIM | TIM4 | Unclear | TIM1, PS | TIMD4 | TIMD4_HUMAN | 1 | | | 0 | Q96H15 |
| IgRSF | CD2/ | CD2 | Stimulatory | CD48 | LFA2, | CD2_HUMAN | 1 | | | 0 | P06729 |
| | SLAM | | | | OX34 | | | | | | |
| IgRSF | CD2/ | SLAM | Stimulatory | SLAM | CD150, | SLAF1_HUMAN | 1 | | | 0 | Q13291 |
| | SLAM | | | | SLAMF1 | | | | | | |
| IgRSF | CD2/ | 2B4 | Stimulatory/ | CD48 | CD244, | CD244_HUMAN | 1 | | | 0 | Q9BZW8 |
| | SLAM | | inhibitory | | SLAMF4 | | | | | | |
| IgRSF | CD2/ | Ly108 | Stimulatory/ | Ly108, | NTBA, | SLAF6_HUMAN | 1 | | | 0 | Q96DU3 |
| | SLAM | | inhibitory | NTB-A | CD352, | | | | | | |
| | | | | (human) | SLAMF6 | | | | | | |
| IgRSF | CD2/ | CD84 | Stimulatory | CD84 | SLAMF5 | SLAF5_HUMAN | 1 | | | 0 | Q9UIB8 |
| | SLAM | | | | | | | | | | |
| IgRSF | CD2/ | Ly9 | Stimulatory | Ly9 | CD229, | LY9_HUMAN | 1 | | | 0 | Q9HBG7 |
| | SLAM | | | | SLAMF3 | | | | | | |
| IgRSF | CD2/ | CRACC | Stimulatory | CRACC | CD319, | SLAF7_HUMAN | 1 | | | 0 | Q9NQ25 |
| | SLAM | | | | BLAME | | | | | | |
| IgRSF | BTN | BTN1 | Unclear | Unknown | BTN1A1 | BT1A1_HUMAN | 1 | | | 0 | Q13410 |
| IgRSF | BTN | BTN2 | Unclear | Unknown | BTN2A1 | BT2A1_HUMAN | 1 | | | 0 | Q7KYR7 |
| IgRSF | BTN | BTN3 | Unclear | Unknown | BTN3A1 | BT3A1_HUMAN | 1 | | | 0 | O00481 |
| IgRSF | LAIR | LAIR1 | Inhibitory | Collagens | | LAIRI_HUMAN | 1 | | | 0 | Q6GTX8 |
| IgRSF | Orphan | LAG3 | Inhibitory | MHCII and | CD223 | LAG3_HUMAN | 1 | | | 0 | P18627 |
| | | | | unknown | | | | | | | |
| IgRSF | Orphan | CD160 | Inhibitory | HVEM | BY55, | BY55_HUMAN | 1 | 27 | 159 | 0 | O95971 |
| | | | | | NK28 | | | | | | |
| TNFRSF | Type-V | 4-1BB | Stimulatory | 4-1BBL | CD137 | TNR9_HUMAN | 1 | | | 0 | Q07011 |
| | (Divergent) | | | | | | | | | | |
| TNFRSF | Type-V | OX40 | Stimulatory | OX40L | CD134 | TNR4_HUMAN | 1 | | | 0 | P43489 |
| | (Divergent) | | | | | | | | | | |
| TNFRSF | Type-V | CD27 | Stimulatory | CD70 | TNFRSF7 | CD27_HUMAN | 1 | | | 0 | P26842 |
| | (Divergent) | | | | | | | | | | |
| TNFRSF | Type-V | GITR | Stimulatory | GITRL | CD357 | TNR18_HUMAN | 1 | | | 0 | Q9Y5U5 |
| | (Divergent) | | | | | | | | | | |
| TNFRSF | Type-V | CD30 | Stimulatory | CD30L | TNFRSF8 | TNR8_HUMAN | 1 | | | 0 | P28908 |
| | (Divergent) | | | | | | | | | | |
| TNFRSF | Type-L | TNFR1 | Apoptosis/ | TNF-_ | CD120a | TNR1A_HUMAN | 1 | | | 0 | P19438 |
| | (Con- | | Survival | | | | | | | | |
| | ventional) | | | | | | | | | | |
| TNFRSF | Type-L | TNFR2 | Apoptosis/ | TNF-_ | CD120b | TNR1B_HUMAN | 1 | | | 0 | P20333 |
| | (Con- | | Survival | | | | | | | | |
| | ventional) | | | | | | | | | | |
| TNFRSF | Type-L | HVEM | Stimulatory | LIGHT, | CD270 | TNR14_HUMAN | 1 | | | 0 | Q92956 |
| | (Con- | | | BTLA, | | | | | | | |
| | ventional) | | | CD160, | | | | | | | |
| | | | | LT_3, | | | | | | | |
| | | | | HSV1gD | | | | | | | |
| TNFRSF | Type-L | LT_R | Stimulatory | LT_1_2, | TNFRSF3 | TNR3_HUMAN | 1 | | | 0 | P36941 |
| | (Con- | | | LIGHT | | | | | | | |
| | ventional) | | | | | | | | | | |
| TNFRSF | Type-L | DR3 | Stimulatory | TL1A | TNFRSF25 | TNR25_HUMAN | 1 | | | 0 | Q93038 |
| | (Con- | | | | | | | | | | |
| | ventional) | | | | | | | | | | |
| TNFRSF | Type-L | DCR3 | | LIGHT, | TNFRSF6B | | 0 | | | 0 | |
| | (Con- | | | TL1A, | | | | | | | |
| | ventional) | | | FASL | | | | | | | |
| TNFRSF | Type-L | FAS | Apoptosis | FASL | CD95 | TNR6_HUMAN | 1 | | | 0 | P25445 |
| | (Con- | | | | | | | | | | |
| | ventional) | | | | | | | | | | |
| TNFRSF | Type-L | CD40 | | CD40L | TNFRSF5 | TNR5_HUMAN | 1 | | | 0 | P25942 |
| | (Con- | | | | | | | | | | |
| | ventional) | | | | | | | | | | |
| TNFRSF | Type-L | RANK | Unclear | RANKL | CD265 | TNR11_HUMAN | 1 | | | 0 | Q9Y6Q6 |
| | (Con- | | | | | | | | | | |
| | ventional) | | | | | | | | | | |
| TNFRSF | Type-L | OPG | Unclear | RANKL | TNFRSF11B | | 0 | | | 0 | |
| | (Con- | | | | | | | | | | |
| | ventional) | | | | | | | | | | |
| TNFRSF | Type-L | TRAILR1 | Apoptosis | TRAIL | TNFRSF10A- | TR10A_HUMAN | 1 | | | 0 | O00220 |
| | (Con- | | | | D | | | | | | |
| | ventional) | | | | | | | | | | |
| TNFRSF | Type-S | TACI | | BAFF, | CD267 | TR13B_HUMAN | 1 | | | 0 | O14836 |
| | (EF- | | | APRIL | | | | | | | |
| | disulfide) | | | | | | | | | | |
| TNFRSF | Type-S | BAFFR | | BAFF | CD268 | TR13C_HUMAN | 1 | | | 0 | Q96RJ3 |
| | (EF- | | | | | | | | | | |
| | disulfide) | | | | | | | | | | |
| TNFRSF | Type-S | BCMA | | BAFF, | CD269 | TNR17_HUMAN | 1 | | | 0 | Q02223 |
| | (EF- | | | APRIL | | | | | | | |
| | disulfide) | | | | | | | | | | |
| TNFRSF | Type-S | TWEAKR | Unclear | TWEAK | CD266 | TNR12_HUMAN | 1 | | | 0 | Q9NP84 |
| | (EF- | | | | | | | | | | |
| | disulfide) | | | | | | | | | | |
| TNFRSF | Type-S | EDAR | Unclear | EDA-A1 | | EDAR_HUMAN | 1 | | | 0 | Q9UNE0 |
| | (EF- | | | | | | | | | | |
| | disulfide) | | | | | | | | | | |
| TNFRSF | Type-S | XEDAR | Unclear | EDA-A2 | TNFRSF27 | TNR27_HUMAN | 1 | | | 0 | Q9HAV5 |
| | (EF- | | | | | | | | | | |
| | disulfide) | | | | | | | | | | |
| TNFRSF | Orphan | RELT | Unclear | Unknown | TNFRSF19L | TR19L_HUMAN | 1 | | | 0 | Q969Z4 |
| TNFRSF | Orphan | DR6 | Inhibitory | Unknown | CD358 | TNR21_HUMAN | 1 | | | 0 | O75509 |
| TNFRSF | Orphan | TROY | Unclear | Unknown | TNFRSF19 | TNR19_HUMAN | 1 | | | 0 | Q9NS68 |
| TNFRSF | Orphan | NGFR | Unclear | Unknown | CD271 | TNR16_HUMAN | 1 | | | 0 | P08138 |
| | | CD22 | | | | CD22_HUMAN | 1 | | | 0 | P20273 |
| | | SIGLEC-3 | | | | CD33_HUMAN | 1 | | | 0 | P20138 |
| | | SIGLEC-5 | | | | SIGL5_HUMAN | 1 | | | 0 | O15389 |
| | | SIGLEC-7 | | | | SIGL7_HUMAN | 1 | | | 0 | Q9Y286 |
| | | KLRG1 | | | | KLRG1_HUMAN | 1 | 0 | 38 | 0 | Q96E93 |
| | | NKR-P1A | | | | KLRB1_HUMAN | 1 | 0 | 45 | 0 | Q12918 |
| | | ILT2 | | | | LIRB1_HUMAN | 1 | | | 0 | Q8NHL6 |
| | | KIR2DL1 | | | | KI2L1_HUMAN | 1 | | | 0 | P43626 |
| | | KIR3DL1 | | | | KI3L1_HUMAN | 1 | | | 0 | P43629 |
| | | CD94-NKG2A | | | | KLRD1_HUMAN | 0 | | | 0 | Q13241 |
| | | CD300b | | | | CLM7_HUMAN | 1 | | | 0 | A8K4G0 |
| | | CD300e | | | | CLM2_HUMAN | 1 | | | 0 | Q496F6 |
| | | TREM1 | | | | TREM1_HUMAN | 1 | | | 0 | Q9NP99 |
| | | TREM2 | | | | TREM2_HUMAN | 1 | | | 0 | Q9NZC2 |
| | | ILT7 | | | | LIRA4_HUMAN | 1 | | | 0 | P59901 |
| | | ILT3 | | | | LIRB4_HUMAN | 1 | | | 0 | Q8NHJ6 |
| | | ILT4 | | | | LIRB2_HUMAN | 1 | | | 0 | Q8N423 |
| | | TLT-1 | | | | TRML1_HUMAN | 1 | | | 0 | Q86YW5 |
| | | CD200R | | | | MO2R1_HUMAN | 1 | | | 0 | Q8TD46 |
| | | CD300a | | | | CLM8_HUMAN | 1 | | | 0 | Q9UGN4 |
| | | CD300f | | | | CLM1_HUMAN | 1 | | | 0 | Q8TDQ1 |
| | | DC-SIGN | | | | CD209_HUMAN | 1 | 0 | 37 | 0 | Q9NNX6 |
| | | B7-2 | | | | CD86_HUMAN | 1 | | | 0 | P42081 |
| | | Allergin-1 | | | | MILR1_HUMAN | 1 | | | 0 | Q7Z6M3 |
| | | LAT | | | | LAT_HUMAN | 1 | | | 0 | O43561 |
| | | BLNK | | | | BLNK_HUMAN | 1 | 0 | 344 | 0 | Q8WV28 |
| | | LAYN | | | | LAYN_HUMAN | 1 | | | 0 | Q6UX15 |
| | | SLP76 | | | | LCP2_HUMAN | 1 | 78 | 420 | 0 | Q13094 |
| | | EBV-LMP1 | | | | LMP1_EBVB9 | 1 | 185 | 386 | 1 | P03230 |
| | | HIV-NEF | | | | NEF_HV1H2 | 1 | 115 | 206 | 1 | P04601 |
| | | HVS-TIP | | | | TIP_SHV2C | 1 | 0 | 228 | 1 | P22575 |
| | | HVS-ORF5 | | | | Q9Q1U5_SHV2 | 1 | | | 1 | Q9Q1U5 |
| | | HVS-stpC | | | | STP_SHV2C | 1 | 0 | 72 | 1 | |

### Nucleic Acids and Vectors

In another aspect, provided herein are nucleic acid molecules comprising the signaling domain oligonucleotide and/or receptor oligonucleotides of the disclosure, including expression cassettes. In yet another aspect, provided herein are expression vectors containing these nucleic acid molecules operably linked to heterologous nucleic acid sequences such as, for example, regulatory sequences which allow in vivo expression of the receptor in a host cell.

The terms “oligonucleotide” and “nucleic acid molecule” are used interchangeably herein as they are in the art and generally refers to polymeric forms of nucleotides of a given length of a given nucleic acid molecule which include sense strand and/or antisense strand. As used herein, the length of oligonucleotide can be about 2 to about 100 nucleotides (nt), about 100 to about 500 nt, about 500 to about 1000 nt, or any length in between. However, oligonucleotides longer than about 1000 nt are also encompassed by the present disclosure. As will be appreciated by one of skilled in the art, oligonucleotide includes deoxyribonucleotides (DNAs), ribonucleotides (RNAs) and their corresponding analogs and derivatives thereof. Oligonucloetides as used herein can be single-stranded or double stranded, and include all formats of chemical modifications or and substitutions, which are both on and in between of nucleotides within a given oligonucleotide. The length of double-stranded oligonucleotides is measured in basepair (bp).

Nucleic acid molecules of the present disclosure can be of any length, including for example, between about 100 bp and about 1 kilobasepair (Kb), between about 1 kilobasepair (Kb) and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

One ordinary skilled in the relevant art would recognize that the chemical modifications and substitutions include, without limitation, chemical modifications or substitutions on the molecular structures of pentose sugar, phosphate group and nitrogenous base of said oligonucleotides. Alternatively oligonucleotides may be labeled with other molecules. In some embodiments, the labeling with other molecules provides a detectable signal, either directly or indirectly. Some non-limiting exemplary labeling methods include fluorescent dyes, biotin, digoxigenin, alkaline phosphatase, and the like. In some aspects, the oligonucleotides are chemically synthesized by methods available in the art.

In some embodiments, the nucleic acid molecules of the present disclosure are incorporated into vectors. In some embodiments, the vector is an expression vector. It will be understood by one skilled in the art that the term “vector” generally refers to a recombinant oligonucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

In some embodiments, the present disclosure encompasses vectors comprising one or more of the signaling domain oligonucleotide disclosed herein. In some embodiments, the present disclosure encompasses a collection of vectors comprising the collection of signaling domain oligonucleotides provided herein. In other embodiments, the present disclosure encompasses a plurality of vectors comprising the plurality of receptor oligonucleotides provided herein.

In some embodiments, the vectors, the collection of vectors, or the plurality of vectors further comprise a plurality of restriction enzyme cleavage sites. In certain embodiments, the restriction enzyme cleavage sites are recognized by 1, 2, or 3 different selected restriction enzymes. In some embodiments, the restriction enzyme cleavage sites are spaced throughout the plasmid no more than about 1,000 bp apart, such that digestion with the selected restriction enzyme(s) produces plasmid fragments no more than about 1,000 bp in length. In certain specific embodiments, the restriction enzyme cleavage sites are spaced throughout the plasmid no more than about 500 bp apart, such that digestion with the selected restriction enzyme(s) produces plasmid fragments no more than about 500 bp in length to facilitate removal of the resulting plasmid fragments via SPRI-based cleanup procedures. Exemplary fragment lengths suitable for the methods and compositions describe herein include plasmid fragments having no more than about 500 bp, no more than about 450 bp, no more than about 400 bp, no more than about 350 bp, no more than about 300 bp, no more than about 250 bp, or no more than about 200 bp in length. However, different fragment lengths can be contemplated by the methods disclosed herein and can be determined according to each specific application.

In certain embodiments, the selected restriction enzymes comprises BsaI, BsmBI, Esp3I, BbsI, NotI, and PspXI. In other embodiments, the vector is a plasmid competent for replication in a host cell.

### Methods of Preparing Receptor Oligonucleotides

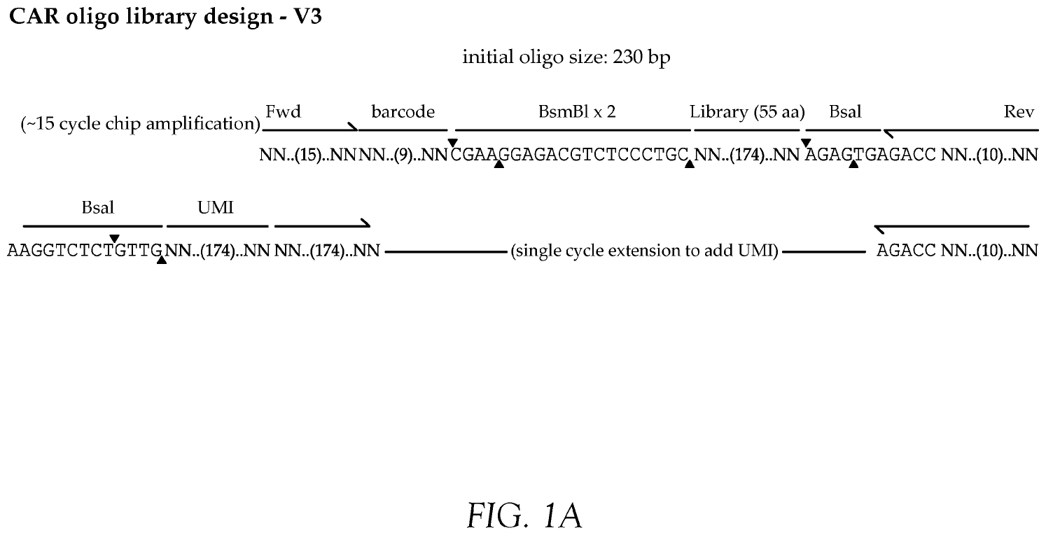

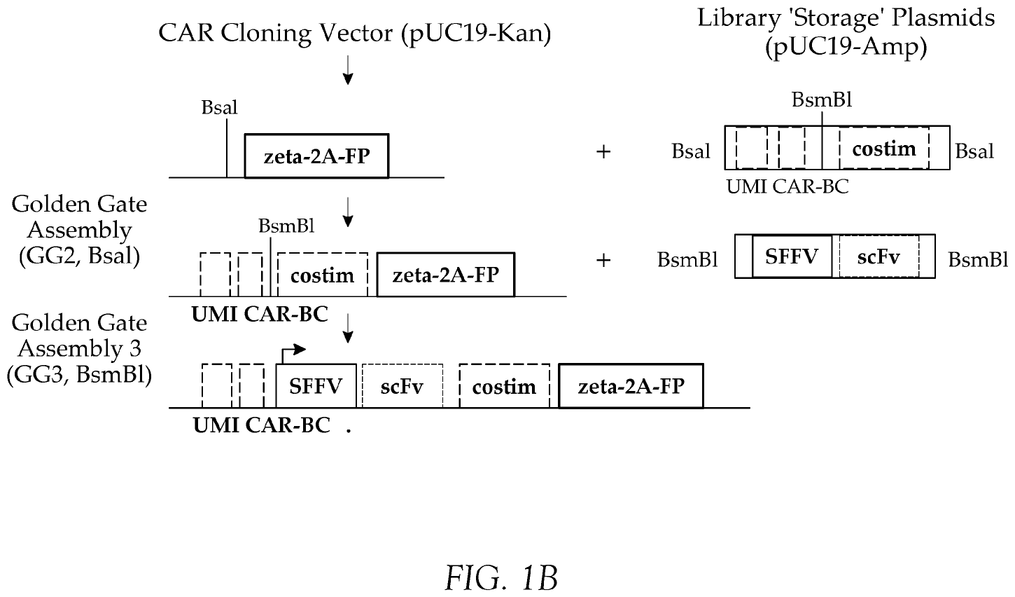

The present disclosure further provides methods for preparing a plurality of receptor oligonucleotides. In some aspects, the methods comprise: a) assembling the collection of signaling domain oligonucleotides provided herein with a plurality of cloning vectors to produce a plurality of intermediate vectors; and b) assembling the plurality of intermediate vectors with a plurality of additional vectors. An exemplary schematic illustration of the method is shown in FIGS. 1A to 1C, FIG. 2, and FIG. 3.

In some embodiments, a member of the plurality of cloning vectors comprises, from 5′ to 3′, one or more of the following components: dual Type IIs restriction sites for a first Type IIs restriction enzyme, an immune cell-activating domain, an autoproteolytic peptide sequence, and a marker protein sequence. In some embodiments, a member of the plurality of cloning vectors comprises, from 5′ to 3′, all of the following components: dual Type IIs restriction sites for a first Type IIs restriction enzyme, an immune cell-activating domain, an autoproteolytic peptide sequence, and a marker protein sequence.

In some embodiments, a member of the plurality of additional vectors comprises one or more of the following components: dual Type IIs restriction sites for a second Type IIs restriction enzyme, an extracellular domain (ECD), sequence and a transmembrane domain. In other embodiments, a member of the plurality of additional vectors comprises all of the following components: dual Type IIs restriction sites for a second Type IIs restriction enzyme, an extracellular domain (ECD), sequence and a transmembrane domain. See, for example, FIGS. 1A to 1C. In certain embodiments, this method is also referred to as the “5′ cloning strategy.”

In some embodiments, a member of the plurality of cloning vectors comprises one or more of the following components: a 5′ homology arm sequence, an extracellular domain (ECD) sequence, a transmembrane domain, dual Type IIs restriction sites for a first Type IIs restriction enzyme, and a 3′ homology arm sequence. In other embodiments, a member of the plurality of cloning vectors comprises all of the following components: a 5′ homology arm sequence, an extracellular domain (ECD) sequence, a transmembrane domain, dual Type IIs restriction sites for a first Type IIs restriction enzyme, and a 3′ homology arm sequence.

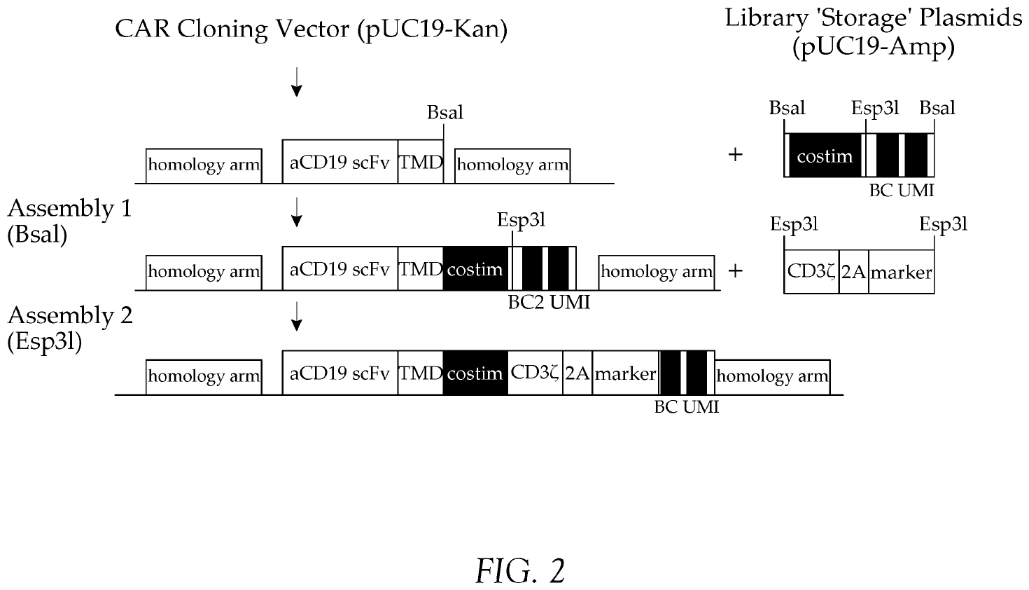

In some embodiments, a member of the plurality of additional vectors comprises one or more of the following components: dual Type IIs restriction sites for a second Type IIs restriction enzyme, an immune cell-activating domain, an autoproteolytic peptide sequence, and a marker protein sequence. In other embodiments, a member of the plurality of cloning vectors comprises all of the following components: dual Type IIs restriction sites for a second Type IIs restriction enzyme, an immune cell-activating domain, an autoproteolytic peptide sequence, and a marker protein sequence. See, for example, FIG. 2 and FIG. 3. In certain embodiments, this method is also referred to as the “3′ cloning strategy,” and is specific to adeno-associated virus (AAV) based cloning strategy.

In some embodiments, the immune cell-activating domain includes one or more immunoreceptor tyrosine-based activation motifs (ITAMs). In some embodiments, the immune cell-activating domain is derived from CD3ζ.

In certain embodiments, the ECD sequence encodes an extracellular antigen-binding domain capable of binding to a target antigen. In some embodiments, the extracellular antigen-binding domain includes an antibody moiety capable of binding to the target antigen. In some embodiments, the antibody moiety is a scFv. Numerous antigen-binding domains are known in the art, including those based on the antigen binding site of an antibody, antibody mimetics, nanobodies, and T-cell receptor fragments. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder such as a DARPin; or a single-chain derived from a T-cell receptor. Accordingly, the antigen-binding domain includes, without limitation, an antibody, a T cell receptor fragment, a soluble T cell receptor, nanobody, aptamer, receptors, fragments or combinations thereof.

In certain embodiments, the antigen-binding domain is a T cell variable region fragments. In other embodiments, the antigen-binding domain is an antibody or fragment thereof. In certain embodiments, the antigen-binding domain is or comprises an antibody or antibody fragment. In certain embodiments, the antibodies are human antibodies, including any known to bind a targeting molecule. The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding domain is a humanized antibody or fragments thereof. A “humanized” antibody is an antibody in which all or substantially all CDR amino acid residues are derived from non-human CDRs and all or substantially all framework region (FR) amino acid residues are derived from human FRs. A humanized antibody optionally may include at least a portion of an antibody constant region derived from a human antibody. A “humanized form” of a non-human antibody, refers to a variant of the non-human antibody that has undergone humanization, in some cases to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In some embodiments, the heavy and light chains of an antibody can be full-length or can be an antigen-binding portion (a Fab, F(ab′)₂, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain”, when used in reference to an antibody, such as an antibody fragment, refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. “Kuby Immunology”, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J Immunol (1993) 150:880-87; Clarkson et al., Nature (1991) 352:624-28.

Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

In certain embodiments, the antigen-binding domain comprises a single chain variable fragment (scFv). As used herein, the term “single-chain variable fragment” or “scFv” comprises a fusion protein of the variable regions of the heavy (V_{H}) and light chains (V_{L}) of an immunoglobulin covalently linked to form a V_{H}::V_{L} heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. In some embodiments, the linker includes glycine for flexibility, and serine or threonine for solubility. Generally, scFv proteins retain the specificity of the original immunoglobulin. Single chain Fv antibodies can be expressed by methods generally well known in the art.

In some embodiments, a member of the plurality of cloning vectors further comprises a promoter sequence or an autoproteolytic peptide sequence. In certain embodiments, the autoproteolytic peptide sequence encodes a 2A self-cleaving peptide. In some embodiments, a member of the plurality of cloning vectors further comprises a marker protein. The marker protein can be included either upstream or downstream of the costimulatory domain, i.e., the signaling domain sequence in the transmembrane domain oligonucleotides described herein. Further, the marker protein can be included either after or before the autoproteolytic peptide sequence. A marker protein as used herein generally refers to a protein that can be used to measure gene expression and generally produces a measurable signal such as fluorescence, color, luminescence, or resistance to a selective reagent. In some embodiments, the marker protein comprises a fluorescent protein. Examples of fluorescent proteins that may be used include, e.g., green fluorescent protein (GFP), eGFP, red fluorescent protein (RFP), blue fluorescent protein (BFP), yellow fluorescent protein (YFP), cyan fluorescent protein (CFP), luciferase, Sirius, Azurite, eBFP2, mTurquoise, eCFP, Cerulean, mTFP1, mUkG1, mAGI, AcGFP, mWasabi, EmGFP, eYFP, Topaz, SYFP2, Venus, Citrine, mKO, mKO2, mOrange, mOrange2, LSSmOrange, PSmOrange, and PSmOrange2, mStrawberry, mRuby, mCherry, mRaspberry, tdTomato, mKate, mKate2, mPlum, mNeptune, T-Sapphire, mAmetrine, mKeima, E2-Orange, E2-Red/Green, and E2-Crimson, ZsGreen. In other embodiments, the marker protein comprises selectable cell surface reporter. Non-limiting examples of selectable cell surface reporters comprise nerve growth factor receptor (NGFR) and a truncated EGF receptor (EGFRt). Other selectable cell surface reporters are known and used in the art and are also contemplated by the present disclosure.

In certain embodiments, one or more of the plurality of receptor oligonucleotides further comprise a 5′ homology arm and a 3′ homology arm.

In some embodiments, the methods further comprise assembling a plurality of vectors comprising guide RNA (gRNA) sequences with the plurality of cloning vectors.

As a general matter on the placement of the barcodes, the closer the barcode is to the corresponding domain or sequence, the barcode shuffling effect in the library (i.e. resulting in an incorrect barcode being associated with a domain) from various sources during PCR or reverse transcription PCR (e.g., if lentivirus is used) is minimized. In one specific embodiment, with lentiviral transduction: the barcode must be placed as close as possible for lentiviral transduction to function efficiently, such as at the 2A self-cleaving peptide between the CAR and the downstream marker gene. In some embodiments, the barcode is placed in noncoding RNA (intronic DNA or UTRs), coding DNA (exons), or outside of the transcript. In certain embodiments, RNA-based readout of the barcode (e.g. amplicon RNA-seq or scRNAseq) is only possible if it is present in the exons or UTRs of the CAR/Marker transcript. In other embodiments, placing the barcode away from exonic or transcribed regions reduces the chance that the barcode itself will impact the function of the library member. In some embodiments, placing the barcode in an intron or in an adjacent Pol III-transcribed region allows the use of an adjacent guide-RNA sequence for combining this method with a CRISPR-screening approach.

In other embodiments, when AAV cloning is used, the placement of the barcodes can be less important. In certain embodiments, the barcode can be put in any of the positions described above, with the described benefits to various placements. Generally, the tradeoff is that placing the barcode closer will reduce recombination, and placing it farther will reduce the impact of the barcode on the CAR function.

In some embodiments of the methods, the assembling steps are conducted concurrently. In certain embodiments, the methods do not use polymerase chain reaction (PCR). In some embodiments, the assembling steps are conducted using Type IIs restriction enzymatic reactions. In contrast, many known methods use PCR-based cloning approaches. However, the disadvantage of using PCR is template switching and reduced product yield. For instance, some methods (e.g., WO2017/040694) use lentivirus and PCR-based cloning strategy. Yet others use PCR and electroporation methods. Thus, to avoid template switching, the methods should avoid using PCR. To achieve this goal, the novel restriction digest strategy provided herein does not involve gel-extraction, ensuring high yield. In particular, in some embodiments, the methods provided herein use backbone digestion using specific enzymes which occur only in the backbone sequence, and not the homology-directed repair (HDR) template, followed by SPRI-based (Solid Phase Reversible Immobilization) size-selection and cleanup as described above, thereby achieving high yield.

In some embodiments, each of the sequences or domains described herein is identifiable by a barcode. In some embodiments of the methods, the assembling steps are conducted combinatorially, for example, see FIG. 4. More specifically, each of the sequences or domains in the signaling domain oligonucleotides and/or any of the vectors used herein is identifiable by a specific barcode. Thus, any of the sequences or domains can be combined with one another to create diversity in the resulting receptor oligonucleotides in a high-throughput manner.

### Receptor Oligonucleotides

Further provided herein are receptor oligonucleotides produced by the methods provided herein. In some embodiments, the present disclosure provides a receptor oligonucleotide comprising, in a 5′ to 3′ direction, one or more of the following components: (a) a unique molecular identifier (UMI), (b) an extracellular antigen-binding domain sequence, (c) a transmembrane domain, (d) a signaling domain sequence, (e) an immune cell-activating domain, and (f) an autoproteolytic peptide sequence. In some embodiments, the receptor oligonucleotide comprises, in a 5′ to 3′ direction, all of the following components: (a) a unique molecular identifier (UMI), (b) an extracellular antigen-binding domain sequence, (c) a transmembrane domain, (d) a signaling domain sequence, (e) an immune cell-activating domain, and (f) an autoproteolytic peptide sequence. In some embodiments, the receptor oligonucleotide further comprises a promoter or an autoproteolytic peptide sequence between the UMI and the ECD sequence. In some embodiments, the receptor oligonucleotide further comprises a gRNA sequence 5′ to the UMI. In some embodiments, the present disclosure provides a plurality, or a library, of receptor oligonucleotides as described immediately above.

In other embodiments, the present disclosure provides a receptor oligonucleotide comprising, in a 5′ to 3′ direction, one or more of the following components: (a) a 5′ homology arm sequence, (b) an extracellular antigen-binding domain (ECD) sequence, (c) a transmembrane domain, (d) a signaling domain sequence, (e) an immune cell-activating domain, (f) an autoproteolytic peptide sequence, (g) a unique molecular identifier (UMI), and (h) a 3′ homology arm sequence. In other embodiments, the receptor oligonucleotide comprises, in a 5′ to 3′ direction, all of the following components: (a) a 5′ homology arm sequence, (b) an extracellular antigen-binding domain sequence, (c) a transmembrane domain, (d) a signaling domain sequence, (e) an immune cell-activating domain, (f) an autoproteolytic peptide sequence, (g) a unique molecular identifier (UMI), and (h) a 3′ homology arm sequence. In some embodiments, the present disclosure provides a plurality, or a library, of receptor oligonucleotides as described immediately above.

In certain embodiments, a receptor oligonucleotide provided herein encodes a chimeric antigen receptor. The term “chimeric antigen receptor” or “CAR” as used herein refers to recombinant receptors that generally contain an extracellular antigen-binding domain and an intracellular signaling domain. In certain embodiments, the CAR also comprises a transmembrane domain. In certain embodiments, the CAR's extracellular antigen-binding domain is composed of a single chain variable fragment (scFv) derived from a fusion protein of the variable regions of the heavy and light chains of an antibody. Alternatively, scFvs may be used that are derived from Fab fragments (instead of from an antibody, e.g., obtained from Fab collection). In various embodiments, the scFv is fused to the transmembrane domain and then to the intracellular signaling domain. “First-generation” CARs include those that solely provide CD3-chain induced signal upon antigen binding. “Second-generation” CARs include those that provide both CD3-chain induced signal upon antigen binding and co-stimulation, such as one including an intracellular signaling domain from a costimulatory receptor (e.g., CD28 or 41BB). “Third-generation” CARs include those that include multiple co-stimulatory domains of different costimulatory receptors. A fourth generation of CAR-T cell includes CAR-T cells redirected for cytokine killing (TRUCK), where the vector containing the CAR construct includes a cytokine cassette. When the CAR-T cell is activated, it deposits a pro-inflammatory or cytotoxic cytokine into the tumor lesion.

### Recombinant Immune Cell

The oligonucleotides of the disclosure can be introduced into a host cell to produce a recombinant or engineered cell containing the oligonucleotides. In some aspects, the terms “recombinant cells” and “engineered cells” are used interchangeably. The present disclosure further comprises recombinant host cells. In some embodiments, the recombinant host cell is a recombinant immune cell. In some embodiments, the recombinant immune cell is capable of expressing a chimeric receptor encoded by the member of the receptor oligonucleotide.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell (T_{H}), a cytotoxic T cell (T_{CTL}), or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+ T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+ T helper lymphocyte cell selected from the group consisting of naïve CD4+ T cells, central memory CD4+ T cells, effector memory CD4+ T cells, and bulk CD4+ T cells. In some embodiments, the cell are obtained by leukapheresis performed on a sample obtained from a subject. In some embodiments, the subject is a human patient.

In some embodiments, the recombinant immune cell is a recombinant T cell. In some embodiments, the recombinant T cell is a recombinant CD4+ T cell or a recombinant CD8+ T cell.

In specific embodiments, the recombinant immune cell is a recombinant T cell and the receptor oligonucleotides encodes a chimeric antigen receptor, thereby producing a CAR-T cell. A CAR-T cell is a T cell that expresses a chimeric antigen receptor.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

Some embodiments of the disclosure relate to methods for making recombinant cells, including the steps of: (a) providing a cell capable of protein expression and (b) contacting the provided cell with one or more members of the plurality of receptor oligonucleotides disclosed herein.

Introduction of the nucleic acid molecules, e.g., the receptor oligonucleotides of the present disclosure, into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, direct micro-injection, nanoparticle-mediated nucleic acid delivery, and the like.

In some embodiments, the nucleic acid molecules are delivered by viral or non-viral delivery vehicles known in the art. For example, the nucleic acid molecule can be stably integrated in the host genome, or can be episomally replicating, or present in the recombinant host cell as a mini-circle expression vector for transient expression. In some embodiments, the nucleic acid molecule is maintained and replicated in the recombinant host cell as an episomal unit. In some embodiments, the nucleic acid molecule is stably integrated into the genome of the recombinant cell. Stable integration can be achieved using classical random genomic recombination techniques, or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant host cell as a mini-circle expression vector for transient expression.

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, the present disclosure further encompasses a collection of the recombinant immune cells (also called “a library”) described herein. Recombinant or engineered cells of the disclosure are useful for studying the properties of different receptors and modulatory signaling domains under different conditions. For instance, the effect of each signaling domain sequence can vary when using T cells obtained from different donors, thus the signaling domain sequence that is best for one donor may be different from the signaling domain sequence that is best for a different donor.

Described herein are methods for making collections of engineered CAR-T cells having a plurality of different signaling domain sequences, and methods for screening such collection for activities and functions under a plurality of different experimental circumstances. In some embodiments, the library comprises a mixture of recombinant or engineered cells, having a plurality of receptor oligonucleotides with different signaling domain sequences. In some embodiments, each of the different signaling domain sequences can be identified with a signaling domain barcode. In some embodiments, the library comprises a collection of engineered cells, having a plurality of receptor oligonucleotides having different signaling domain sequences. In some embodiments, the plurality of receptor oligonucleotides comprises 2, 3, 4, 5, 6, 7, 8, 9, 10, 12, 15, 20, 25, 30, 35, 40, 41, 42, 43, 44, 45, 46, 47, 48, 49, or about 50 different signaling domain sequences. In certain embodiment, each member of the collection of the recombinant immune cells is identifiable by a UMI.

In some embodiments, the recombinant immune cell exhibits one or more improved properties including, without being limited to: cellular proliferation; resistance to immune cell exhaustion; expression of cytokines; sensitivity to antigen; specificity to antigen; stability of differentiation state (e.g. T-regulatory cells); trafficking to a specific tissue or organ in vivo; ability to kill target cells; or differentiation into a desired state.

### Screening Methods

The present disclosure further encompasses methods for identifying, or screening, a signaling domain sequence that modulates a designated property of a cell under specified conditions. In some embodiments, the method generally comprises: providing the recombinant immune cell or the collection of recombinant immune cells disclosed herein; applying one or more specified conditions to the recombinant immune cell or the collection of recombinant immune cells; identifying the recombinant cells that exhibit the designated property; and/or identifying the signaling domain sequence in the recombinant cells that exhibit the designated property.

As described in more detail below, in certain embodiments, the specified conditions comprise interaction with a target antigen. In certain embodiments, the designated property comprises one or more of the following: cellular proliferation; resistance to immune cell exhaustion; expression of cytokines; sensitivity to antigen; specificity to antigen; stability of differentiation state; trafficking to a specific tissue or organ; ability to kill target cells; or differentiation into a desired state. In certain embodiments, the identification is performed in vitro or in vivo. In certain embodiments, wherein the identification can be performed by techniques commonly used in the art, such as, without limitation, single-cell sequencing, fluorescence-activated cell sorting (FACS), bead-based enrichment, enrichment in a population of mixed cells over time after a stimulus, and the methods described below.

There are many parameters of CAR-T cell function and performance, for example, the cell's ability to proliferate in response to its target antigen, its ability to resist exhaustion and survival, its ability to survive despite constant or repeated stimulation, its ability to kill target cells, its ability to secrete cytokines, its ability to ignore immunosuppressive target cell tactics, its ability to differentiate into memory cells, its ability to avoid damaging non-target cells, its ability to survive in a tumor microenvironment, and others. There are also many factors that can affect the ability of a CAR-T cell to perform its functions, such as the genetics and state of the T cells from which the CAR-T cell is generated (e.g., individual genetic variation from donor to donor); the particular characteristics of the target cells, such as the expression level of the target antigen, expression or secretion of immune checkpoint molecules, or resistance to cytokines or cytotoxins; the characteristics of the target cells environment, such as acidity, hypoxia, and physical barriers, and others. Additionally, there are factors regarding therapy that can affect the response of CAR-T cells, such as concurrent immunotherapy, chemotherapy, radiation, and other medications that the subject may require. The effect of each of these parameters, factors, situations, and conditions can be examined using a library of the disclosure in a suitably designed experiment.

CAR-T cell experiments often use target cells in order to stimulate proliferation, differentiation, cytokine expression, cytotoxicity, and/or exhaustion. Target cells can be any type of cell capable of expressing the target antigen (the antigen against which the CAR is directed), and need not be viable. Model cell lines are often selected to simulate the cells to be targeted during treatment, for example using a lymphoma cell line to test receptor oligonucleotides intended for treating lymphoma. The target cells can also be obtained from a subject to be treated, for example tumor cells or leukemia cells from a subject to be treated can be obtained, for example, by biopsy or blood draw respectively. Alternatively, CAR-T cells can be stimulated using beads having suitable antigens or antibodies bound to the surface, for example having anti-CD3 and anti-CD28 antibodies bound to the surface.

### I. Proliferation

T cells expand clonally in response to antigen-specific stimulation through the T cell receptor, in combination with co-stimulation from other factors. Without being bound by any particular theory, it is believed that costimulation by proteins such as CD28 and 4-1BB are necessary for efficient clonal expansion, and that including the signaling domain(s) from such proteins in a receptor oligonucleotide results in comparable activation and proliferation. This clonal expansion is a necessary part of the immune response, which insures that enough T cells are generated to combat the target cells as the number of target cells increases. However, T cells should not proliferate in the absence of such stimulation. The target cells are in general either normal cells infected with a virus or other intracellular pathogen, or are neoplastic or cancerous cells. Thus, proliferation is an important measure of CAR-T cell function and utility.

Proliferation can be measured by a number of different methods, depending on the type of library used. For example, where the library is a collection of different CAR-T clones (each clone having a different signaling domain sequence), each clone can be examined in individual cultures in parallel. In such cases, the number of CAR-T cells can be measured or estimated at times during the experiment and/or at the end of the experiment, for example without limitation by sorting and counting the cells (e.g., by FACS), and/or by quantifying the amount of signal from a marker protein such as green fluorescent protein (GFP), mCherry, mTomato, or the like. In the case of mixture collection, CAR-T cells can also be sorted and counted in the same manner, if the library is constructed with a different marker protein for each different clone. This is practical as long as each marker protein can be distinguished from the other marker proteins in the sorting device, which generally limits such mixture collection to about 4 to 7 different clones. For larger mixture collection (i.e., mixture collection comprising about 7 to about 60 different clones), other methods of counting are more practical, such as single cell sequencing. For such mixture collection, the receptor oligonucleotide can be provided with primer sequences that facilitate sequencing the signaling domain sequence and/or the entire receptor. In some embodiments, the library is a mixture library, and the proliferation of individual clones within the library is determined by single cell sequencing.

### II. Exhaustion

T cells can become exhausted and/or anergic in response to constant or frequently repeated stimulation, at which point they lose the ability to proliferate, express inflammatory cytokines, and kill target cells. Exhausted T cells can be characterized by the elevated expression of surface proteins such as TIM3, PD-1, LAG3, CTLA-4, CD39, CD43 and CD69, and reduced expression of proteins such as CD62L and CD127. It is accordingly a goal to identify receptor oligonucleotides that help T cells to resist exhaustion.

Resistance to exhaustion can be measured in appropriately designed experiments, for example, by measuring the clonal expansion rate of a library (e.g., using the methods for measuring proliferation above) in response to constant stimulation for at least a period of time sufficient to cause exhaustion in normal T cells, and determining when (or if) the rate of proliferation declines or ceases. Libraries can also be subjected to cytokines that induce exhaustion, such as IL-10 or TGFβ, and assayed for any reduction in the rate or amount of exhaustion induced. Alternatively, collection can be tested by sorting and measuring clones for expression of exhaustion markers, such as, for example, TIM3, PD-1, LAG3, CTLA-4, CD39, CD43 and CD69. In some embodiments, a library is subjected to conditions that can cause anergy, and is labeled for detection of one or more of the exhaustion markers TIM3, PD-1, LAG3, CTLA-4, CD39, CD43 and CD69. In some embodiments, the library is labeled for detection of 1, 2, 3, 4, 5, 6, or 7 exhaustion markers. Expression of one or more exhaustion markers can alternatively be measured by other means, for example using single cell RNA sequencing (scRNAseq). In some embodiments, a library is subjected to conditions that can cause anergy, and the expression of one or more of the exhaustion markers TIM3, PD-1, LAG3, CTLA-4, CD39, CD43 and CD69 is determined by scRNAseq. In some embodiments, the expression of 1, 2, 3, 4, 5, 6, or 7 exhaustion markers is determined by scRNAseq. In some embodiments, the library is a collection library. In some embodiments, the library is a mixture library. In some embodiments, the exhaustion marker is TIM3, PD-1, LAG3, or CD39. In some embodiments, the exhaustion markers are TIM3, PD-1, LAG3, and CD39.

Various methods can be used to measure T cell exhaustion, including without limitation, FACS. As a general matter, FACS-based measurements can be extended beyond single fluorescent channels (e.g., Cell Trace Violet or intracellular stains) to multiparameter gatings to extract data on T cell subsets or combinations of exhaustion markers.

### III. Differentiation

After a T cell has been fully activated by exposure to antigen and the appropriate costimulatory factors, it may further differentiate into a memory T cell. A memory T cell can remain in the body for decades, and responds to the antigen it recognizes more quickly and strongly than a naïve T cell. Accordingly, it is useful to identify costimulatory domains that increase or accelerate differentiation of CAR-T cells into memory CAR-T cells.

Memory T cells are usually identified by the expression of characteristic surface protein markers, such as CD45RO. Different subclasses of memory T cells can also be identified by the expression of characteristic surface protein markers. Central memory T cells (T_{CM}, CD45RA⁺ CD45RO⁺ CCR7⁺ CD62L⁺) are found mainly in lymph nodes and in the peripheral circulation. Effector memory (T_{EM}, CD45RA⁻ CD45RO⁺ CCR7⁻), and effector memory re-expressing CD45RA (T_{EMRA}, CD45RA⁺ CD45RO⁺ CCR7⁻) cells are found mainly in the peripheral circulation and in tissues. Naïve T cells generally display CD45RA⁺ CD45RO⁺ CCR7⁺ antigens. In some embodiments, the library is stained for CD45RO expression after stimulation, and the number of CD45RO⁺ cells is quantified. In some embodiments, the library is examined for CD45RO expression by scRNAseq, and the number of CD45RO⁺ cells is quantified. In some embodiments, the library is stained for CD45RO expression after stimulation, and the number of CD45RO⁺ cells in a plurality of different clones is quantified. In some embodiments, the library is examined for CD45RO expression by scRNAseq, and the number of CD45RO⁺ cells in a plurality of different clones is quantified. In some embodiments, the library is examined for CD45RO expression at a number of time points after initial stimulation with antigen. In some embodiments, the library is examined at no less than 1, 2, 3, 4, 5, 6, 7, 8, 9, or 10 different time points after initial stimulation with antigen. In some embodiments, the library is examined at no more than 50, 40, 30, 20, 15, 14, 13, 12, 11, 10, 9, 8, 7, 6, or 5 different time points after initial stimulation with antigen. In some embodiments, the library is a collection library. In some embodiments, the library is a mixture library.

### IV. Cytotoxicity

The ability to kill target cells is a primary function of T cells, particularly CD8+ T cells. CD4+ T cells can also exhibit direct cytotoxic and anti-viral effects through the secretion of perform, granzyme B, and/or IFNγ. As set forth herein, different signaling domain sequences can stimulate cytotoxicity to differing degrees. As with the other receptor oligonucleotide parameters, one goal is to find signaling domain sequences that highly increase antigen-induced cytotoxicity. Another goal is to find signaling domain sequences that produce little or no tonic cytotoxicity (i.e., cytotoxicity in the absence of the target antigen). Another goal is to find signaling domain sequences that simultaneously satisfy the first two goals.

Cytotoxicity can be measured using the standard protocols known in the art. In general, cytotoxicity is measured in a collection library, so that cytotoxicity can be attributed to the correct receptor oligonucleotide. In general, living target cells are contacted with a library of engineered cells, followed by determination of target cell survival or death. Depending on the target cells, cytotoxicity may result in target cell death, or only a reduction in target cell proliferation (as compared to suitable controls). Target cell death can be quantified, for example, using a vital stain, which is excluded from target cells having an intact membrane, but enters damaged cells to stain intracellular structures. Examples of vital stains include Trypan Blue, 7AAD (7-aminoactinomycin-D), propidium iodide, Zombie Green™ (and other Zombie stains, BioLegend, San Diego, CA). Alternatively, target cells can be transduced with a marker protein such as mKate, mCherry, GFP, and death of the target cells can be determined by reduction in the marker protein signal. Using such methods, cytotoxicity can be determined at one or more time points, or continuously. In some embodiments, the death of target cells is determined continuously.

### V. Cytokines

CD4 T cells also respond to antigen stimulation by expressing and releasing cytokines such as interleukin-2 (IL-2), tumor necrosis factor-α (TNFα), and gamma interferon (IFNγ), which further increase immune functions. IL-2 induces T cells to differentiate into effector T cells and memory T cells. TNFα stimulates inflammatory responses. IFNγ has antiviral, antibacterial, and antifungal activity, and stimulates the differentiation of CD4+ T_{H} cells into T_{H}1 T cells. In some embodiments, a library of engineered cells is stimulated by contacting it with an antigen, and expression of at least one of IL-2, TNFα, and IFNγ is measured. In some embodiments, expression of two or more of IL-2, TNFα, and IFNγ is measured. In some embodiments, expression of IL-2, TNFα, and IFNγ is measured.

Cytokine expression can be measured directly, using commercially available immunoassays such as ELISA antibody-based kits. Intracellular cytokines can be stained by permeabilizing cells, and adding antibodies specific for the cytokines to be detected. If a mixture library is used, the cells can be sorted and identified (e.g., by single cell sequencing) after staining. Alternatively, cytokine expression can be measured using techniques such as scRNAseq. In some embodiments, a library of engineered cells is stimulated by contacting it with an antigen, and expression of at least one of IL-2, TNFα, and IFNγ is measured by immunoassay. In some embodiments, a library of engineered cells is stimulated by contacting it with an antigen, and expression of at least one of IL-2, TNFα, and IFNγ is measured by scRNAseq.

### Systems and Kits

Also provided herein are systems and kits including the signaling domain oligonucleotides or a collection thereof, receptor oligonucleotides, recombinant cells, or vector compositions provided and described herein as well as written instructions for making and using the same. For example, provided herein, in some embodiments, are systems and/or kits that include one or more of: a collection of signaling domain oligonucleotides as described herein, a plurality of receptor oligonucleotides as described herein, a recombinant cell as described herein, or a vector, a collection of vectors, or a plurality of vectors as described herein.

In some embodiments, the systems and/or kits of the disclosure further include one or more syringes (including pre-filled syringes) and/or catheters (including pre-filled syringes) used to administer one any of the provided chimeric receptors, recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cancer cell, or treating a disease in an individual in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the chimeric receptor polypeptides.

In some embodiments, a system or kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

Throughout this specification, various patents, patent applications and other types of publications (e.g., journal articles, electronic database entries, etc.) are referenced. All patents, patent applications, and other publications cited in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferre, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

### Example 1

### Computational Design of Costimulatory Signaling Domains and Domain Fragments for Testing

This example illustrates the computational design of the signaling domain oligonucleotide. The process comprises the following steps.

### 1. Obtain Signaling Domain Sequences

A number of different sequence categories were tested. MYSQL and SPARQL queries with custom Python scripts, across several computational databases, were used to identify following: (1) human proteins in UniProt categorized as class I or class III membrane proteins (Set A); (2) proteins for a list of manually annotated known costimulatory proteins from human and a few from human-associated viruses (Set B) including: CD28, ICOS, CTLA4, PD1, PD1H, BTLA, B71, B7H1, CD226, CRTAM, TIGIT, CD96, TIM1, TIM2, TIM3, TIM4, CD2, SLAM, 2B4, Ly108, CD84, Ly9, CRACC, BTN1, BTN2, BTN3, LAIR1, LAG3, CD160, 4-1BB, OX40, CD27, GITR, CD30, TNFR1, TNFR2, HVEM, LT_R, DR3, DCR3, FAS, CD40, RANK, OPG, TRAILR1, TACI, BAFFR, BCMA, TWEAKR, EDAR, XEDAR, RELT, DR6, TROY, NGFR, CD22, SIGLEC-3, SIGLEC-5, SIGLEC-7, KLRG1, NKR-P1A, ILT2, KIR2DL1, KIR3DL1, CD94-NKG2A, CD300b, CD300e, TREM1, TREM2, ILT7, ILT3, ILT4, TLT-1, CD200R, CD300a, CD300f, DC-SIGN, B7-2, Allergin-1, LAT, BLNK, LAYN, SLP76, EMB-LMP1, HIV-NEF, HVS-TIP, HVS-ORF5, and HVS-stpC; (3) Proteins of particular interest (a subset of set B) for even more in-depth study based on their costimulatory function: OX40, ICOS, 4-1BB, CTLA4, CD28, CD30, CD2, CD27, and CD226 (Set C); (4) annotated sequences in UniProt and in the Eukaryotic Linear Motif Database (ELM) contained within the proteins above; (5) annotated or predicted phosphorylation or ubiquitination sites in NETPHOS and PhosphoSite within the proteins above; (6) transcripts for the proteins above classified as the primary gene product; (7) UniProt-annotated transmembrane region from each of the above proteins; (8) for proteins in Set A or Set B with known receptor tyrosine kinase domains annotated by UniProt, those domains were identified and removed with the goal to leave only the linear sequence regions of the proteins behind; (9) a landscape of amino acid conservation for each residue of each transmembrane region using UNICLUST multiple sequence alignments; (10) the human nucleotide sequences for each of the transmembrane region using CCDS and ENA; and (11) a list of all missense mutations, which occur in the extracted regions of the domains above, using ENSEMBL ProtVar.

### 2. Generate Sequence Variants Based on the Above Protein Domains.

Sequence variants based on the above protein domains were generated. For different sets, different methods were used to generate costimulatory sequences to synthesize and test.

To facilitate pooled synthesis using the Agilent 230-bp synthesis, all oligonucleotides synthesized had to be 230 base pairs at most, which, after accounting for cloning regions, barcodes, and priming sites, is 55 amino acids. In cases where the resulting domain is larger than this, it was separated into overlapping chunks with an overlap of 22 amino acids between adjacent chunks. Domains shorter than 15 amino acids were discarded due to potential issues with PCR bias. Domains longer than 440 amino acids from Set A were also not considered.

For phosphorylation and ubiquitination knockouts, versions of domains with all ubiquitinated or all phosphorylated residues replaced with alanine were generated. For deletion scan, variant chunks of all chunks for all proteins in Set B and C were generated by changing sliding windows of 6 amino acids, moving 3 amino acids at a time, to alanine. For constitutive phosphorylation mimics, variant chunks of all chunks of domains in sets B and C were generated where all tyrosine (Y), serine (S), and threonine (T) residues annotated to be phosphorylated were changed to aspartic acid (D). For known human variations, versions of the proteins, which contain the known human missense mutations from ENSEMBL ProtVar as described in above, were made. For deep mutational scan, all single amino acid insertions, deletions, and changes, one group per domain were generated for all proteins in set C.

### 3. Generate Novel Chimeric Signaling Domain Sequences.

Chimeric signaling domain sequences, each consisting of two chunks, were generated. Each chunk was generated from following 3 sources:
1) Proteins from Set A containing the following domains, as annotated by UniProt, Box 1, ITIM, ITSM, interaction with GRB2, interaction with SRC and ESR1, LIR, NPXY, PDZ-binding, PDZ-binding (in isoform 2), PPPSP, PPXY, PPxY, PTB-like, SH2-binding, SH3-binding, or SLAM-like.
2) Proteins from Set A containing the following domains, as annotated by ELM, SH2, SH3, MAPK, WIRS, TRAF, ITAM, ITSM, ITIM, PTB, PDZ, Actin, PP2A, or PKB. Only annotated domains with known experimental evidence were selected, but not those with only computational predictions.
3) Regions of 5-30 base pairs in a subset of curated costimulatory domains that manually were chosen for a combination of increased local amino acid conservation with UNICLUST and the annotation of predicted or known costimulatory or coinhibitory domains from UniProt and ELM.

These chunks were shuffled together into pairs to be synthesized as chimeric signaling domain sequences. Chunks were randomly paired so that each chunk was in both the first and second position two times each. Each chunk must also occur in total at least 6 times. The final average size of the chimeric signaling domain is between about 25 to about 50 amino acids. However, shorter or longer chimeric signaling domain can be obtained depending on the synthesizing platform used.

### 4. Groupings of Sequence Variants

Sequence variants based on the above rules were grouped as following:
1. Group 0—‘min_vip’: Domains from Set C, wildtype versions only;
2. Group 1—‘all_vip’: Domains from Set C with wildtype sequence and deletion scans;
3. Group 2—‘curated’: Domains from Set B and Set C, with variants from all the categories listed in the above Sequence Variant section;
4. Group 3—‘all_wt’: Domains from Set A, wildtype versions only;
5. Group 4—‘ko_curated’: Domains from Set B and Set C, wildtype, phosphorylation knockouts, and ubiquitination knockouts;
6. Group 5—‘wt_curated’: Domains from Set B and Set C, wildtype versions only;
7. Group 6—‘chimeric’ from Novel Chimeric Domain section above;
8. Group 7—One group for each domain from Set C: all single amino acid insertions, deletions, and changes, one group per domain.

Each group of the sequence variants can be pulled by the common primer sequences that are shared among the variants in each group.

### 5. Generation of Orthogonal Barcodes for Every Sequence in Every Group.

The entire library was synthesized in two different ways for two types of cloning systems, and therefore each group was represented twice in the oligo pool, once for each cloning system. For these two cloning systems, two types of oligo barcodes were generated: noncoding and coding. In this example, both noncoding and coding barcodes were 9 base pairs in length. However, barcodes with various lengths may be used. A person skilled in the art would understand how to optimize the length of the barcodes as needed.

For noncoding barcodes, 240,000 25-mer barcodes were generated using this algorithm described in Qikai Xu, et al. (Design of 240,000 orthogonal 25mer DNA barcode probes; Proceedings of the National Academy of Sciences; January 2009, pnas.0812506106; DOI: 10.1073/pnas.0812506106). The longer barcodes were split into 9 base pairs sub-barcodes, and they were further checked for uniqueness and the absence of restriction sites. For coding barcodes, all possible 3-codon triples that are not stop codons or the bulky aromatic amino acids tryptophan, phenylalanine, and tyrosine, were generated. Barcodes that have greater than or equal to 3 consecutive G or C nucleotides or greater than or equal to 4 consecutive A or T nucleotides were removed. Barcodes that have a GC content greater than 70% or less than 30% were also removed. Finally, barcodes with self-complementarity of greater than or equal to 2 base pairs were removed.

6. Generation of Signaling Domain Oligonucleotides Based on these Sequence Variants and Barcodes.

Based on above sequence variants and barcodes, signaling domain oligonucleotides were generated. Sets of primer pairs with an average length of about 15 base pairs were generated using a method which optimized overlaps, primer dimers, and secondary structure. See Sriram Kosuri et al. (Nat Biotechnol. 2010 December; 28(12): 1295-1299). The melting temperature for each signaling domain oligonucleotide was kept close to 60° C. under the desired annealing conditions. Each set above had a unique pair of PCR priming sites that allowed it to be amplified independently off of a single chip-synthesized oligonucleotide pool. To conserve base pairs on each oligonucleotide, a portion of one of the primers contained a portion of the external BsaI restriction site, GGTCT. Specifically, the reverse primer for the 3′ cloning system and the forward primer for the 5′ cloning system contained this sequence.

### 7. Requirements for Signaling Domain Oligonucleotide Sequence.

First, the signaling domain oligonucleotides must contain Type IIS restriction site regions to allow for Golden Gate assembly. For example, some oligonucleotides include the internal BsmBI restriction sites, which were used to insert an arbitrary sequence between the barcode and the costimulatory domain, i.e., the signaling domain sequence in the transmembrane domain oligonucleotides. An overlapping pair of BsmBI restriction sites was designed to conserve space on the oligonucleotides, containing two outward facing BsmbI sites: AGAGGGAGACGTCTCCGCCT (SEQ ID NO: 1).

Next, the oligonucleotides must not contain any of the restriction sites used for plasmid assembly. For instance, the restriction sites used for plasmid assembly in this Example, were BsaI (both orientations), BsmBI (both orientations), BbsI (both orientations), NotI, and PspXI (including all possible variants). Thus, the oligonucleotide sequences were screened to avoid the creation of the aforementioned restriction sites, both within the domain codon sequence itself and at the junctions created between barcode, internal restriction site, external restriction sites, and PCR priming sites. Sequences were further screened for close annealing matches to the PCR priming sites. In cases where restriction sites were created, codons were changed synonymously using a weighted replacement algorithm based on the human codon usage table.

### 8. The 3′ and 5′ Cloning Systems

A Unique Molecular Identifier (UMI) is a random sequence that was not part of the signaling domain oligonucleotide sequence but was added with a final cycle of PCR as an additional spiked-in sequence. This UMI allowed each individual oligonucleotide molecule to have a unique identifying sequence, allowing to distinguish and track individual transduction, transfection, or integration events across the T cell library once it has been inserted into T cells.

In the 5′ cloning system, the barcode was placed upstream of the promoter, so that the promoter, ScFv, and transmembrane regions of the chimeric signaling domain sequence were inserted into the internal restriction site region. The schematic illustrations are shown in FIGS. 1A-1C.

In the 3′ cloning system, the barcode was placed downstream of the chimeric signaling domain sequence, so that the CD3 zeta domain, the 2A peptide, the marker protein, and a final polyA/2A peptide/feature barcode were inserted into the internal restriction site region. The schematic illustration is shown in FIG. 2. In this illustration, Esp3I was an isoschizomer of BsmbI, and only one potential scheme of barcode/UMI addition (2A/Marker/barcode+UMI) was shown. However, additional schemes, including without being limited to, 2A/Marker/barcode+UMI+FBC/2A, 2A/Marker/2A/barcode+UMI+FBC, 2A/Marker/barcode+UMI+FBC/polyA, may also be used. FBC refers to feature barcode, which generally is a defined sequence used to anneal and identify the CAR barcode region via the 10x Genomics scRNAseq platform.

### Example 2

### Amplification of the Library from Chip-Synthesized DNA

### 1. Library Amplification

Each sub-library, i.e., the groups 0-7 above, was amplified separately off of the chip DNA to limit potential amplification bias and conserve the limited resource of chip-synthesized DNA. Each sub-library had a separate primer set. The DNA concentration and number of PCR cycles for each sub-library were empirically determined, using more template for smaller sub-libraries to limit cycles and amplification bias. Exemplary library amplification components are shown in Table 2. Exemplary library amplification conditions are shown in Table 3.

In this Example, between about 5 and about 70 pM of template was used. An average of 20 PCR cycles were used. The UMI primers were added at the final cycle during a pause in the extension step.

After amplification, individual sub-libraries were examined on Agarose gel to confirm size and purity, and subsequently SPRI-purified at a 1.8× ratio of SPRI to DNA to remove small products. Finally, each linear sub-library was cloned at library efficiency using Lucigen electrocompetent cells into an Ampicillin-marked storage plasmid so it could be propagated and re-used. The aim was colony count of >1×10⁷/reaction per library to carry forward as many UMIs as possible. Library efficiency refers to E. coli transformation efficiency. Generally, ‘library efficiency’ of transformation means >10⁸ CFUs per μg of plasmid DNA. However, the numbers vary. For instance, Lucigen SUPREME electrocompetent cells used in these experiments have 10¹⁰ CFUs per μg.

TABLE 2
Library Amplification Components

| Component | Per 50 μL reaction |
|---|---|
| Kapa HIFI (2x) | 45 |
| Fwd Primer (250 nM) | 2.25 |
| Rev Primer (250 nM) | 2.25 |
| Template | 20 |
| H₂O | 30.5 |

TABLE 3
Library Amplification Conditions

| Step | Time | | Temp | Cycles |
|---|---|---|---|---|
| Hotstart | 3 | min | 95 | |
| Denat | 20 | sec | 98 | 20 cycles |
| Anneal | 15 | sec | 60 | |
| Extend | 20 | sec | 72 | |

Add 2.5 μL of primer mix and perform one final cycle as above.

| | | |
|---|---|---|
| Final Extend | 3 | 72 |

### 2. Construction of an AAV Backbone Vector

A custom AAV backbone vector was built based on the sequence published by Eyquem et al., Nature 543:113-117 (2017). A kanamcyin resistance marker was used, and undesired restriction sites were removed. Extra restriction sites (NotI and PspxI) internal to the 3′ and 5′ Inverted Terminal Repeats (ITRs) were inserted in the vector in order to swap homology arm and the chimeric signaling domain constructs in and out of the backbone easily without requiring PCR amplification of the ITRs themselves.

Optimized backbone versions were made for each of the various chimeric signaling domain and marker layouts by varying the use and placement of the final 2A sequence, the presence of a polyA which truncates the TRAC transcript, and the location of the UMI and 10X Genomics feature barcode sequences, either upstream of a final 2A attached to the marker, downstream of a final 2A, or before a polyA sequence.

3. Cloning of the Amplified DNA Library after Amplification

Two successive Golden Gate reactions were used to generate the library plasmids from the amplified linear DNA. The cloning can generally be performed using a Golden Gate standard protocol. The Golden Gate protocol can also be optimized by one skilled in the art according to use.

BsaI was used in the first Golden Gate reaction to insert the library plasmid (Ampicillin) into a plasmid (Kanamycin) containing either an AAV plasmid backbone or a simple cloning plasmid for extraction later in the case of non-viral integration. The plasmid contained different elements depending on the 5′ or the 3′ cloning scheme, as described in FIGS. 1A-1B and FIG. 2, respectively. A 3:1 vector to insert mass ratio was used (10 ng/μL; 50 μL reaction for each library and 125 ng of insert and 325 ng of backbone). The components of the first Golden Gate reaction are shown in Table 3. Because the backbone plasmid had Kanamycin resistance and the library plasmid had Ampicillin resistance, background in the double-plasmid Golden Gate protocol could be avoided. The libraries in 10 μL, 125 ng were distributed. Initial cutting was with BbsI, and PCR tubes were put in the thermocycler at 37° C. for 30 minutes. 1 μL T4 Ligase and 5.5 μL of 10 mM ATP (final concentration 1 mM) were added. The thermocycler cycle protocol was as follows: (1) 10 minutes at 16° C.; (2) 5 minutes at 37° C.; (3) return to step (1) for a total of 10 cycles; (4) 15 minutes at 37° C.; and (5) 5 minutes at 80° C. At the end of the cycle, exonuclease mix (7 μL 10 mM DTT, 1 μL Exonuclase V (RecBCD), and 7 μL 10 mM ATP) was added to remove linear DNA that remained. The reaction was put back on thermocycler for 30 minutes at 37° C. followed by 5 minutes at 80° C. Finally, SPRI was selected at 1:1 and each was eluted into 25 μL.

TABLE 3
Components of the First Golden Gate Cloning Protocol

| Component | 1x | |
|---|---|---|
| Cutsmart buffer | 5 | μL |
| BbsI-HF | 0.5 | μL |
| Backbone plasmid | 375 | ng |
| library insert | 125 | ng |
| DI H₂O | to 50 | μL |

BsmbI/Esp3I was used in the second Golden Gate reaction to insert the constant region in between the CAR and the barcode. The sequence of this insertion varied between the 3′ and 5′ methods, as depicted schematically in FIGS. 1A-1B and FIG. 2, respectively. The constant region was on an Amp backbone plasmid so the double-plasmid Golden Gate protocol could be performed. The components of the first Golden Gate reaction are shown in Table 4. Using a similar protocol to the one above for the first Golden Gate reaction, the reaction was run in the thermocycler for 24 cycles: 45° C. 2 minutes; 20° C. 4 minutes; 50° C. 60 minutes; 80° C. 10 minutes; and kept at 12° C. Finally SPRI was cleaned up at 1:1 and the resulting sample was eluted in a small volume (5 μL) to prepare for electroporation.

TABLE 4
Components of the Second Golden Gate Cloning Protocol

| Component | 1x | |
|---|---|---|
| ~75 fmol backbone (product of first golden gate) | 275 | ng |
| ~150 fmol insert plasmid | 500 | fg |
| T4 DNA ligase buffer | 5 | |
| T4 DNA ligase (2 × 10⁶ U/mL) | 0.5 | |
| Esp3I (NEB) | 2 | |
| H₂O | to 50 | μL |

For electroporation, 1 μL of the eluted DNA was added to 25 μL of Lucigen E. Cloni Supreme cells. A colony count in excess of 1×10⁷ per cuvette reaction was expected. To estimate Transformant count, colony counts were checked using dilution series plating of 10 μL, and total transformants were back-calculated with a Poisson sampling model. The remaining volume of transformed cells were grown out for 1 hour with shaking at 30° C. and plated onto 2 large square Petri plates per sample. After 24 hours of growth, or confluence on the plate, the culture was scraped off carefully and diluted back to a reasonable OD. Aliquots were made for glycerol storage and the remainder was maxi-prepped.

4. Plasmid Preparation for AAV Vs. Non-Viral Integration

For AAV, after maxi-prepping, plasmid could be used directly for generation of AAV virions using standard protocols. For non-viral integration, plasmid DNA could either be used directly in the electroporation (see below) or the backbone could be removed and the DNA payload linearized with the following method: PspXI and NotI restriction sites were inserted into a Kanamycin backbone at various sites determined by bioinformatic and empirical analyses to have no effect on plasmid propagation or selection. These restriction sites allowed the backbone to be cut into fragments less than about 500 base pairs in size. After digestion with PspXI and NotI, the library plasmid backbone fragments were removed with a modified SPRI reaction, where the SPRI bead reagent is diluted 1:1 with TE. These diluted beads were then combined at 1:1 ratio with the digestion reaction, resulting in the removal of the small backbone fragments and leaving behind the linear DNA for electroporation. Libraries were then checked for fidelity with a set of diagnostic restriction enzyme digests and by spot-checking approximately 6-12 colonies by Sanger Sequencing.

### 5. Preparation of Primary T Cells for Receipt of the Library

PBMCs were prepared from a TRIMA residual using the standard RosettaSep/SepMate protocol. PBMCs were either aliquotted and frozen down in liquid nitrogen using standard cryostorage media and protocols, or used directly. Bulk T cells were prepared from the PBMCs using the Stem Cell CD3 Isolation Kit and standard protocols. If CD4s and CD8s were used separately, then the Stem Cell CD4 and CD8 isolation kits were used on separate aliquots of PBMCs.

6. Non-Viral Integration or AAV-Based Integration of the Library into Primary T Cells

After isolation of T cells, Cas9, guide RNA were electroporated to make a cut upstream/at the 5′ edge of exon 6 of the TRAC locus. The following guide sequences were used to cut, depending on the homology arms used:

(SEQ ID NO: 2)
AGAGTCTCTCAGCTGGTACA
(SEQ ID NO: 3)
CAGGGTTCTGGATATCTGT
(SEQ ID NO: 4)
CTGGATATCTGTGGGACAAG

Cells were plated at 10⁶/mL in X-Vivo media plus 5% human AB serum, 30-100 U of IL2, and 10 mM neutralized N-acetyl L-Cysteine. CD3/CD28 T cell expansion beads (DynaBeads) were added at the standard concentration, either 4-24 hours after T cell thaw for frozen T cell aliquots, or directly after isolation with fresh PBMCs/T cells. After 48 hours on beads, beads were removed by magnetic separation. Electroporation was performed 0 to 24 hours after bead removal.

The protocol was similar to the protocol published by Roth et al. Nature 559:405-09 (2018). First trRNA was mixed with crRNA at a 1:1 ratio at 37° C., and then NLS-tagged Cas9 and a PGA polymer (Nyugen et al. bioRxiv. 27 Mar. 2019) were added along with the linear or plasmid library DNA, in the case of non-viral integration. T cells were spun down and resuspended in Lonza P3 electroporation media at 4° C. so that 5×10⁵ to 2×10⁶ cells were resuspended in 20 μL. The total volume of cells, RNA, polymer, DNA, and Cas9 was 22-25 μL per well of a Lonza 96-well electroporation cuvette plate.

Electroporation used method E115 in the Lonza Nucleofector software and cells were recovered immediately in the same X-Vivo media as described above without IL2. After 30 minutes of recovery, cells were counted and resuspended from 10⁶ to 3×10⁶ cells/mL in X Vivo media with 30-100 U IL2.

For AAV, the procedure was the same as above except the DNA was not added during the electroporation step. Instead, AAV virions were added at an MOI of 10⁵-10⁶ virions/cell and left for 18-24 hours. After 24 hours, cells were resuspended in fresh media to remove the virus.

During expansion after electroporation, cells were replated every 1-3 days to keep the total cell density at approximately 10⁶ cells/mL.

### 7. Staining of the Library for Assessment of Transduction/Transfection Efficiency

To stain for loss of CD3 and integration of the CAR, cells were stained with CD3, Myc, NGFR (if using an NGFR marker), and Zombie Yellow. In the case of bulk T cells, CD4 and/or CD8 was also stained to compare the ratio of CD4 cells to CD8 cells, and the difference in transfection efficiency between the two T cell types.

8. Downstream Assays with the Library

Details of these assays for stimulation timing, staining, and sorting conditions can be optimized by a person of ordinary skill in the art based on specific application of the methods provided herein. In some exemplary aspects, genomic DNA was extracted from the sorted cells using the Machery Nagel 96-well, S or XS genomic DNA extraction kits. The barcode region was amplified using primers designed for that purpose. A second PCR was performed using barcoded per-sample Illumina Primers, as is standard for multiplexed Illumina sequencing.

While this disclosure has been particularly shown and described with references to preferred embodiments thereof, it will be understood by those skilled in the art that various changes in form and details may be made therein without departing from the scope of the disclosure encompassed by the appended claims.

1. A signaling domain oligonucleotide, comprising from 5′ to 3′:
a) a Type IIs restriction site for a first Type IIs restriction enzyme;
b) a forward primer sequence;
c) a module selected from the group consisting of:
i) a module comprising a signaling domain barcode, dual Type IIs restriction sites for a second Type IIs restriction enzyme, and a signaling domain sequence; and
ii) a module comprising a signaling domain sequence, dual Type IIs restriction sites for a second Type IIs restriction enzyme, and a signaling domain barcode;
d) an additional Type IIs restriction site for the first Type IIs restriction enzyme; and
e) a reverse primer sequence;
wherein at least one of the forward primer sequence and the reverse primer sequence comprises at least one Type IIs restriction site;
wherein the Type IIs restriction sites for the first Type IIs restriction enzyme and the second Type IIs restriction enzyme are not identical.

2. The signaling domain oligonucleotide of claim 1, wherein signaling domain barcode does not contain a restriction site for either the first Type IIs restriction enzyme or the second Type IIs restriction enzyme.

3. The signaling domain oligonucleotide of claim 2, wherein the signaling domain barcode:
a) indicates the identity of the signaling domain sequence;
b) comprises no more than about 3 consecutive G or C bases;
c) comprises no more than about 4 consecutive A or T bases; and/or
d) comprises a GC content that is at least about 30% and no more than about 70%.

4. The signaling domain oligonucleotide of claim 1, wherein the signaling domain sequence does not contain a restriction site for either the first Type IIs restriction enzyme or the second Type IIs restriction enzyme.

5. The signaling domain oligonucleotide of claim 1, wherein the signaling domain sequence comprises one or more sequences each independently derived from a sequence encoding an intracellular portion of a membrane protein.

6. The signaling domain oligonucleotide of claim 5, wherein each of the one or more sequences in the signaling domain sequence is at least 80% identical to the sequence encoding the intracellular portion of the membrane protein.

7. The signaling domain oligonucleotide of claim 5, wherein the membrane protein comprises a class 1 human membrane protein, a class 3 human membrane protein, or a membrane protein from a species that is capable of infecting a mammalian cell.

8. The signaling domain oligonucleotide of claim 5, wherein the membrane protein is selected from the list consisting of CD28, ICOS, CTLA4, PD1, PD1H, BTLA, B71, B7H1, CD226, CRTAM, TIGIT, CD96, TIM1, TIM2, TIM3, TIM4, CD2, SLAM, 2B4, Ly108, CD84, Ly9, CRACC, BTN1, BTN2, BTN3, LAIR1, LAG3, CD160, 4-1BB, OX40, CD27, GITR, CD30, TNFR1, TNFR2, HVEM, LT_R, DR3, DCR3, FAS, CD40, RANK, OPG, TRAILR1, TACI, BAFFR, BCMA, TWEAKR, EDAR, XEDAR, RELT, DR6, TROY, NGFR, CD22, SIGLEC-3, SIGLEC-5, SIGLEC-7, KLRG1, NKR-P1A, ILT2, KIR2DL1, KIR3DL1, CD94-NKG2A, CD300b, CD300e, TREM1, TREM2, ILT7, ILT3, ILT4, TLT-1, CD200R, CD300a, CD300f, DC-SIGN, B7-2, Allergin-1, LAT, BLNK, LAYN, SLP76, EMB-LMP1, HIV-NEF, HVS-TIP, HVS-ORF5, and HVS-stpC.

9. (canceled)

10. The signaling domain oligonucleotide of claim 1, wherein the signaling domain sequence comprises more than one sequence, each derived from a sequence encoding an intracellular portion of a different membrane protein.

11. The signaling domain oligonucleotide of claim 1, wherein the signaling domain sequence comprises one or more mutations relative to a wild-type sequence.

12. The signaling domain oligonucleotide of claim 11, wherein the one or more mutations comprise phosphorylation and ubiquitination knockouts, deletion scans, constitutive phosphorylation mimics, known human mutations, and/or deep mutational scan.

13. The signaling domain oligonucleotide of claim 1, wherein (i) the signaling domain sequence encodes a peptide that can no longer be modified by phosphorylation or ubiquitination; and/or (ii) all codons for tyrosine, serine, and threonine residues in the signaling domain sequence are replaced with codons for aspartic acid.

14. (canceled)

15. The signaling domain oligonucleotide of claim 1, wherein the first Type IIs restriction enzyme and the second Type IIs restriction enzyme are independently selected from the group consisting of BsaI, BsmBI, Esp3I, BbsI, NotI, and PspXI.

16. (canceled)

17. The signaling domain oligonucleotide of claim 1, further comprising a Unique Molecular Identifier (UMI).

18. A collection of signaling domain oligonucleotides comprising a plurality of the signaling domain oligonucleotides of claim 1, wherein each one of the signaling domain oligonucleotides has a unique pair of the forward primer sequence and the reverse primer sequence.

19. A method for preparing a plurality of receptor oligonucleotides, comprising:
a) assembling the collection of signaling domain oligonucleotides of claim 18 with a plurality of cloning vectors to produce a plurality of intermediate vectors, wherein a member of the plurality of cloning vectors comprises, from 5′ to 3′, dual Type IIs restriction sites for a first Type IIs restriction enzyme, an immune cell-activating domain, an autoproteolytic peptide sequence, and a marker protein sequence; and
b) assembling the plurality of intermediate vectors with a plurality of additional vectors, wherein a member of the plurality of additional vectors comprises dual Type IIs restriction sites for a second Type IIs restriction enzyme, an extracellular domain (ECD), sequence and a transmembrane domain,
thereby producing the plurality of receptor oligonucleotides.

20. A method for preparing a plurality of receptor oligonucleotides, comprising:
a) assembling the collection of signaling domain oligonucleotides of claim 18 with a plurality of cloning vectors to produce a plurality of intermediate vectors, wherein a member of the plurality of cloning vectors comprises, a 5′ homology arm sequence, an extracellular domain (ECD) sequence, a transmembrane domain, dual Type IIs restriction sites for a first Type IIs restriction enzyme, and a 3′ homology arm sequence;
b) assembling the plurality of intermediate vectors with a plurality of additional vectors, wherein a member of the plurality of additional vectors comprises dual Type IIs restriction sites for a second Type IIs restriction enzyme, an immune cell-activating domain, an autoproteolytic peptide sequence, and a marker protein sequence;
thereby producing the plurality of receptor oligonucleotides.

21-31. (canceled)

32. A plurality of receptor oligonucleotides produced by the method of claim 19.

33-37. (canceled)

38. A vector comprising the signaling domain oligonucleotide of claim 1.

39-44. (canceled)

45. A recombinant immune cell, comprising one or more of the plurality of receptor oligonucleotides of claim 32, wherein the recombinant immune cell is capable of expressing a chimeric receptor encoded by the receptor oligonucleotide.

46-55. (canceled)
